(19) United States
(12) Patent Application Publication
(10) Pub. No.: US 2012/0226132 A1
(43) Pub. Date: Sep. 6, 2012

(54) IMPLANTABLE OPHTHALMIC MEMS SENSOR DEVICES AND METHODS FOR EYE SURGERY

(75) Inventors: Vernon G. Wong, Menlo Park, CA (US); Douglas A. Lee, Menlo Park, CA (US)

(73) Assignee: OrthoMEMS, Inc., Menlo Park, CA (US)

(21) Appl. No.: 13/414,063

(22) Filed: Mar. 7, 2012

Related U.S. Application Data
(63) Continuation of application No. PCT/US2010/049527, filed on Sep. 20, 2010.
(60) Provisional application No. 61/243,843, filed on Sep. 18, 2009.
(60) Provisional application No. 61/335,572, filed on Jan. 8, 2010.

(51) Int. Cl. A61B 3/16

(52) U.S. Cl. 600/398

(57) ABSTRACT

Methods and apparatus for measurement of IOP following glaucoma surgery comprise an implant device having a pressure sensitive capacitor and coil sized for placement along the tissue drainage path, to monitor the success of the surgery and measure IOP directly. The implantable sensor device may comprise a MEMS based capacitive pressure sensor and coil. A complaint material is disposed over the pressure sensitive capacitor and coil to conform with tissue to further decrease invasiveness and such that the implant can measure pressure from at least a first side and a second side when positioned along the drainage path. The implant can work well with trabeculectomies and trabeculotomies, and can be positioned on the sclera at a location corresponding to the bleb, such that the effectiveness of the surgery and medication can be determined postoperatively to detect pressure changes and elevations.

### CROSS-REFERENCES TO RELATED APPLICATIONS

The present application is a continuation of PCT Application No. PCT/US/2010/049527, filed on Sep. 20, 2010, which claims priority to the following patent applications: U.S. Pat. App. Ser. No. 61/243,843 filed on Sep. 18, 2009, entitled “Implantable Ophthalmic MEMs Sensor Devices and Methods”, and U.S. Pat. App. Ser. No. 61/335,572, filed on 8 Jan. 2010, entitled, “Implantable Ophthalmic MEMs Sensor Devices and Methods for Eye Surgery”, the full disclosures of which are incorporated herein by reference.

### BACKGROUND OF THE INVENTION

People like to see. The eye is a complex organ that allows a person to see his or her surroundings. The eye includes a cornea and crystalline lens that form an image on the retina of the eye. The retina of the eye senses the light image formed thereon and transmits neural signals via the optic nerve to the occipital cortex of the brain, such that the person can see and perceive his or her surroundings. Unfortunately, ocular diseases can compromise vision of the eye and may cause blindness in at least some instances.

Glaucoma is a major cause of blindness in the United States. In many instances, glaucoma related blindness can be prevented if caught and managed early. Glaucoma is usually associated with an increase in intraocular pressure (hereinafter “IOP”), that can result in damage to the retina of the eye. Because glaucoma is usually associated with an increase in IOP, periodic testing can be used to monitor glaucoma in order to prevent irreversible vision loss. For example, a person may undergo two to four exams per year in an ophthalmologist's office, although more examination may sometimes occur. In at least some instances patient compliance is less than ideal, as can miss patients scheduled appointments, such that the IOP is not measured in a timely manner and appropriate therapy and treatment may be delayed. Although treatment can be effective in many instances, in at least some patients may continue to lose vision under physician directed care. For example, about fifteen percent of patients under fifty years of age may continue to lose vision when receiving care and about thirty percent of patient over sixty may continue to lose vision.

Although measurements with an external IOP sensor can be helpful, these devices that Measure pressure of the eye with an external sensor are somewhat indirect and can be inaccurate in at least some instances, such that the measured IOP may differ from the actual pressure inside the eye. In at least some instances, clinically available IOP sensors determine the IOP based on the externally measured pressure. For example, the IOP sensor can measure pressure of the eye on the external surface of the cornea, for example with applanation or indentation of the cornea. The externally sensed pressure of the eye can be used to determine the IOP of the eye based on assumptions about the anatomy and characteristics of the patient's eye. In at least some instances, such assumptions can lead to errors in the indirectly measured IOP when the anatomy of the patient deviates from the assumed normal anatomy and assumed normal characteristics of the eye. Consequently, a patient may not receive appropriate treatment in at least some instances.

A significant clinical need exists for treatment of glaucoma patients with medically uncontrolled IOP and vision loss from optic nerve damage (i.e., worse cases). Many approaches to the treatment of glaucoma are directed to improving drainage of the eye so as to lower IOP. Although drugs can be effective in lowering IOP, treatment with drugs may not be sufficient or affordable, such that at least some level of surgical intervention may be required in at least some instances. Although the surgical insertion of shunts can be used to treat glaucoma, shunts can extend through tissue structures of the eye so as to be somewhat more invasive than would be ideal in at least some instances. Also, at least some shunts can be rigid, such that the eye can be somewhat more vulnerable to trauma in at least some instances. Consequently, shunts may be considered as one of the last options available to at least some patients for the treatment of glaucoma in at least some instances.

One approach to the treatment of glaucoma is for an ophthalmic surgeon to perform surgery such as a trabeculectomy or a trabeculotomy. For example, a trabeculectomy can be performed so as to improve drainage of the eye and lower IOP. Many people have received trabeculectomies to successfully lower IOP. For example, total trabeculectomies in 2006 (with or without previous surgery or trauma) that Medicare reimbursed were approximately 52,500 cases. This number excludes a significant number of U.S. patients with glaucoma aged 40 to 64 who are covered by private insurance.

Although trabeculectomies and other surgeries such as trabeculotomy can be successful in lowering IOP, in at least some instances the surgical outcome is less than ideal. Scar tissue can form so as to decrease drainage of the eye, for example so as to decrease drainage through the drainage hole. Patients can undergo examination regularly, and such examinations may not detect the formation of scar tissue as quickly as would be ideal. Also, IOP spikes and rapid changes in IOP that may cause neural damage may go undetected, such that a patient may continue to lose at least some visual acuity after surgery in at least some instances.

In light of the above, it would be helpful to provide methods and apparatus that overcome at least some of the above shortcomings of glaucoma treatment, for example so as to provide direct measurement of IOP at least daily following glaucoma surgery and in a manner that is less invasive than current devices.

### BRIEF SUMMARY OF THE INVENTION

Embodiments of the present invention provide methods and apparatus for improved glaucoma surgery and improved measurements of IOP following the glaucoma surgery. The apparatus may comprise an implant device having a transducer coupled to transmission circuitry, such as a pressure sensitive capacitor and a coil, respectively, each sized such that the implant can be used as an adjunct to surgery. The implantable sensor device can be sized such that the implanted sensor can be positioned at a location along a surgically created drainage path, for example between the sclera and conjunctiva, so as to monitor the outcome of the surgery and decrease invasiveness of the implant. As the implant can be localized to scleral tissue or positioned between the sclera and conjunctiva, sensitivity of the eye to trauma can be decreased. The implant may comprise a MEMS based capacitive pressure sensor and coil, such that the implant can be sized to decrease invasiveness. Also, the implant may comprise a complaint material disposed over the pressure sensitive capacitor and coil to conform with tissue to further decrease invasiveness. The implant device with the complaint material can measure pressure at many locations of the sensor, for example pressure from locations along a 360 degree perimeter and from at least a first side and a second side, which can improve measurements when the sensor positioned between layers of tissue. The implant can work well with trabeculectomies and trabeculotomies, and can be positioned at many locations along the surgically generated tissue drainage path, for example within a drainage bleb formed between the conjunctiva and sclera, or under a scleral flap between the flap and bed. The effectiveness of the surgery can be determined postoperatively on at least a daily interval, for example hourly, so as to detect pressure spikes and closure of the surgically formed drainage channel based on measurements from the sensor located along the drainage path. The liquid of the anterior chamber can be coupled to the sensor along the drainage path, such that the sensor implant may provide a direct measurement of IOP.

### BRIEF DESCRIPTION OF THE DRAWINGS

FIGS. 1A to 1C show an eye suitable for incorporation with an implantable sensor, in accordance with embodiments of the present invention;

FIG. 2A shows implantation of a sensor along a surgically generated drainage path, for example of a trabeculectomy, in accordance with embodiments;

FIGS. 2B1 to 2B3 show a sensor implanted under conjunctiva of an eye and under a tissue flap of the eye to measure IOP as in FIG. 2A, in accordance with embodiments of the present invention;

FIGS. 2C1 to 2C3 show placement of a sensor as in FIG. 2B1 with a trabeculectomy, in which the sensor positioned on the sclera away from the flap and bed and such that the sensor is located within the bleb when the bleb forms with separation between the sclera and conjunctiva;

FIGS. 2D1 to 2D3 show a placement of a sensor as in FIG. 2B1 in which the sensor is positioned with a trabeculotomy on the sclera such that the sensor is located within the bleb when the bleb forms with separation between the sclera and conjunctiva;

FIG. 2E shows locations and surgical placement of the implantable sensor in accordance with embodiments;

FIG. 3 shows components of a telemetry system comprising the implantable sensor, in accordance with embodiments of the present invention;

FIG. 3A shows components of an implantable sensor as in FIGS. 2A and 2B, in accordance with embodiments of the present invention;

FIG. 3A1 shows the implantable sensor having a coil comprising a substantially single loop antenna and a second coil;

FIG. 3A2 shows the implantable sensor comprising a MEMS pressure sensor on a flexible support;

FIG. 3B shows packaging of an implantable sensor, in accordance with embodiments of the present invention;

FIG. 3C shows a delivery tool for placement of an implantable sensor as in FIG. 2A, in accordance with embodiments of the present invention;

FIG. 4A shows components of an antenna reader, in accordance with embodiments of the present invention;

FIG. 4B shows a hand held antenna reader with components similar to the antenna reader as in 4A;

FIG. 4C shows a docking station to receive the hand held antenna reader as in 4B;

FIG. 5 shows a method of treating and monitoring a patient, in accordance with embodiments of the present invention;

FIG. 6A shows an implantable sensor package for direct measurement of IOP prior to placement in an eye of a rabbit, in accordance with embodiments of the present invention;

FIG. 6B shows the experimentally tested implantable sensor as in FIG. 6A implanted in the anterior chamber of an eye of a rabbit;

FIG. 6C shows a rabbit positioned near an antenna reader with the sensor implanted as in FIGS. 6A and 6B;

FIG. 6D shows a distribution of sensor signals and a peak of the distribution for IOP measured directly with the rabbit positioned near the sensor as in FIG. 6C;

FIG. 6E shows pressure shifts of IOP measured directly over time with the rabbit positioned near the sensor reader as in FIG. 6C; and

FIG. 6F shows IOP measured directly over time with calibration for the rabbit positioned near the sensor reader as in FIG. 6C.

### DETAILED DESCRIPTION OF THE INVENTION

Embodiments described herein will have application to surgery to reduce intraocular pressure, for example with trabeculectomy and trabeculotomy. Although specific reference is trabeculectomy filtering surgery, there are new variations to this surgical procedure that can benefit in accordance with embodiments described herein. For example, the methods and apparatus described herein can be used with surgeries that involve forming a hole under the conjunctiva, a bleb, a scleral flap, and a channel communicating with the anterior chamber, such that aqueous fluid can drain from the anterior chamber, thereby lowering IOP. Also, the systems, methods and devices as described herein can be used as an adjunct with many surgeries such as retinal surgery and cataract surgery, and the implant can be positioned at many locations of the eye to directly measure IOP, for example one or more of intracorneal, anterior chamber, anterior segment, posterior chamber, posterior segment, vitreous and vitreous cavity, sub-retinal space, suprachoroid, suprachoroidal space, subconjunctiva, episcleral, intrascleral, periocular, trabeculotomy sites, trabeculectomy sites, or cyclodialysis space.

The implantable sensor device can be positioned at many locations of glaucoma surgery that correspond to a drainage path of the glaucoma surgery, such that the success of the surgery and drainage along the path can be monitored, and such that the intraocular pressure can be monitored. For example, the implantable sensor device can be used with each of trabeculectomies and trabeculotomies, and can be positioned in the bleb that forms between the sclera and conjunctiva, along a surgically generated drainage channel, or within a bed under a flap of sclera, for example.

Many embodiments described herein provide a direct measurement of intraocular pressure. The intraocular pressure can be measured as often as practical, for example with a hand held reader coupled to the implanted device. The measurements can be made with sufficient frequency so as to determine the presence of diurnal IOP curves and so as to detect IOP peaks and pressure spikes. For example, the measurements can be generated hourly for the first few days following surgery, and then with decreasing frequency as the patient's pressure stabilizes. The hand held device may automatically forward the patient information to the treating physician, such that the physician can monitor the patient remotely.

As used herein, the anterior segment of the eye encompasses the anterior chamber of the eye and the posterior chamber of the eye.

As used herein, trabeculectomy encompasses a surgery that includes separating a flap of sclera from an underlying bed and forming a channel extending from the bed to the to the anterior chamber so as to the treat glaucoma.

As used herein, trabeculotomy encompasses a surgery similar trabeculectomy that forms a surgical channel extending from the upper sclera to the anterior chamber so as to the treat glaucoma.

As used herein, Tenon's capsule encompasses an outer layer of the sclera of the eye.

The implantable sensor device may comprise fenestrations, or holes, so as to allow the device to be anchored to tissue at a desired location with sutures.

Embodiments of the present invention utilizes MEMS and wireless technology that can provide direct, continuous and real-time data on IOP. The embodiments can also provide IOP data and measurements as often as practical and desirable to treat the patient.

The implantable portion of the system comprises a miniature, battery-less, wireless pressure sensor that can be implanted in the under the conjunctiva, for example under the conjunctiva separated from the sclera to from a bleb or within the sclera beneath the partial-thickness scleral flap created during glaucoma filtering surgery (GFS) such as trabeculectomy.

In the embodiments with trabeculotomy, the sensor is implanted under the conjunctiva near or under the bleb next to the filtration pathway.

Using a custom delivery tool, the sensor implant can be placed next to or on top of the surgically created opening or hole that communicates with the anterior chamber. The implant device can be anchored to the sclera, for example with sutures that extend through fenestrations of the implant device and into scleral tissue. Once sutured and closed, the scleral flap can completely cover the implantable device and secures the implantable device from migration outside the scleral flap.

The implant can be removed, for example in case of adverse events.

Following implantation, direct IOP measurements can be obtained real-time and continuously with a data acquisition unit that wirelessly interrogates the implanted sensor, and includes hardware/software to control an external antenna and monitor pressure fluctuation patterns for normal/pathological conditions. The IOP measurement comprises a direct measurement as the transducer of the pressure sensor is implanted along the surgically created tissue drainage path such that the transducer is fluidically coupled to a target tissue structure of interest. For example, the transducer can be coupled to the anterior chamber with the fluid that drains along the drainage path so as to provide a direct measurement of IOP.

The direct IOP measurement data can be used in many beneficial ways. For example, the direct IOP measurement data can be used to trigger an alarm for the patient with the hand held reader, and the data can be transmitted to a remote server and to the office of the treating physician. The data at the remote server can be analyzed, for example mined, to determine statistical trends and analysis and algorithm development. The algorithm can be embodied in instructions of a computer program of the server. The data at the physician's office can be used by the physician to monitor the patient.

The implantable MEMS pressure sensor device and external telemetry may include telemetry components as described in U.S. Pat. Nos. 6,706,005, 6,682,490, and 6,447,449, the full disclosures of which are incorporated by reference and suitable for combination in accordance with some embodiments of the present invention described herein.

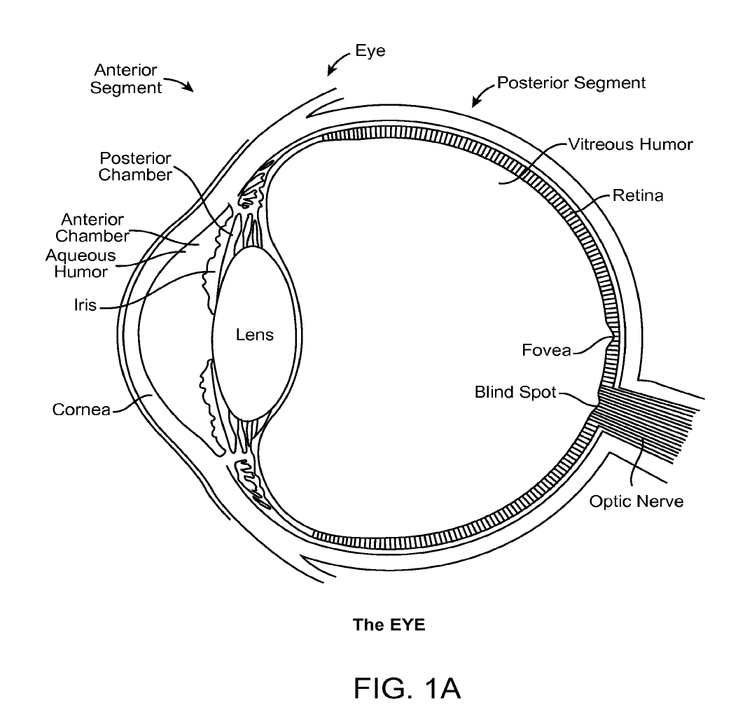

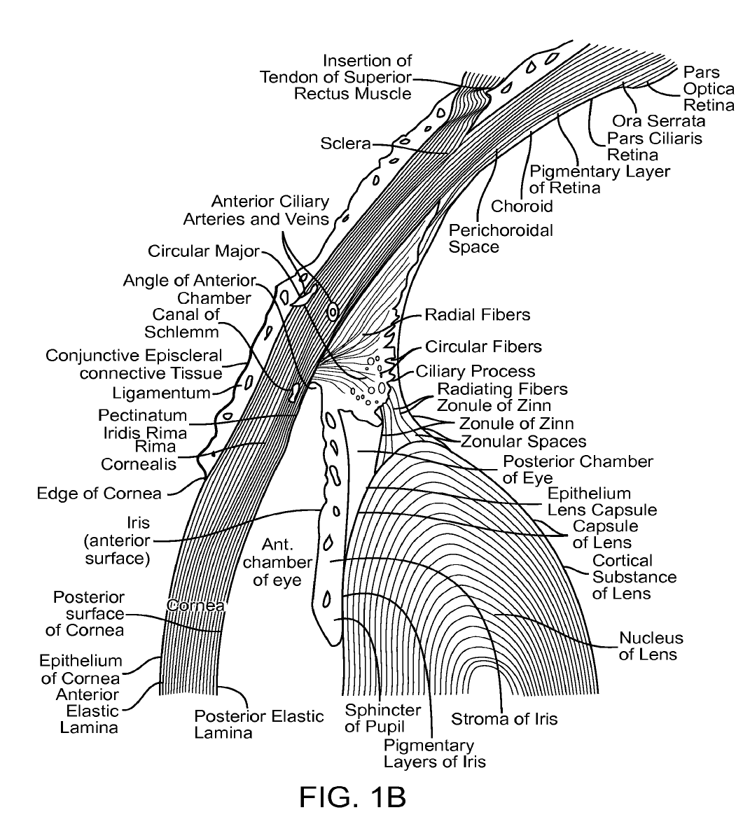

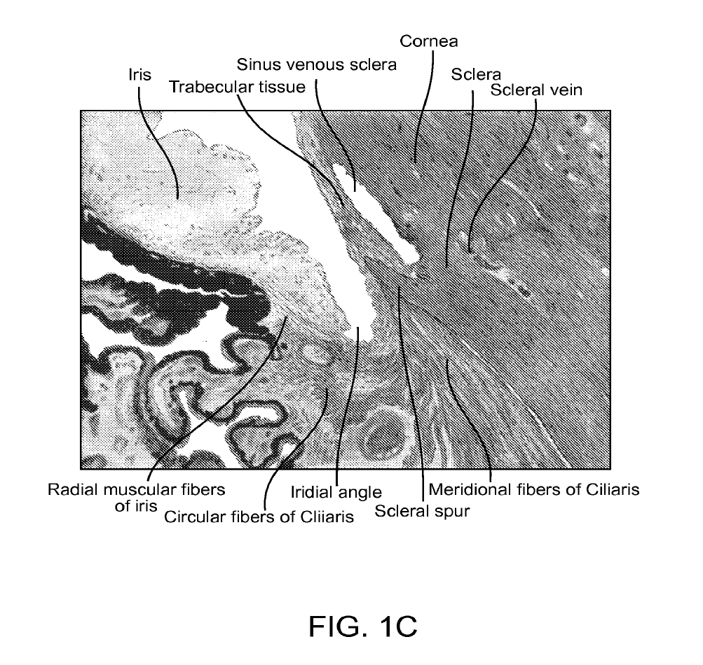

FIGS. 1A to 1C show an eye suitable for incorporation with an implantable sensor, and figures similar to FIGS. 1B and 1C can be found in Grey's Anatomy and available on the Internet, for example at the online encyclopedia Wikipedia (http://www.en.wikipedia.com). The eye comprises a cornea and lens that refract light so as to form an image on the retina of the eye. The retina comprises a fovea comprising light sensitive cones to detect light color sensitivity and high visual acuity. The retina also comprise a blind spot where the optic nerve couples to the retina. An iris is disposed over the lens and responds to light so as to dilate in darkness and constrict in bright light, such that the intensity of light striking the retina can be increased and decreased, respectively. The eye comprises an anterior segment and a posterior segment, with the lens disposed therebetween. The anterior segment comprises an aqueous humor and the posterior segment comprises a vitreous humor. The posterior chamber of the eye extends between the iris and the anterior capsule of the lens and comprises the aqueous humor. The anterior segment comprises the posterior chamber. The liquid of the eye generally drains from the posterior segment to the anterior segment and out Schlemm's canal so as to maintain intraocular pressure.

Schlemm's canal, also known as canal of Schlemm or the scleral venous sinus, comprises a circular channel in the eye that collects aqueous humor from the anterior chamber and delivers the liquid of the aqueous humor into the bloodstream. The canal comprises an endothelium-lined tube. On the inside of the canal, nearest to the aqueous humor, the canal is covered by the trabecular meshwork, and this region contributes to outflow resistance of the aqueous humor.

With glaucoma, the drainage of aqueous liquid from the anterior chamber is less than ideal such that pressure in the anterior chamber increases. As the liquid of the anterior chamber communicates with the posterior chamber, the pressure of the whole eye is increased, including the back of the eye comprising the retina, such that the retina can be damaged.

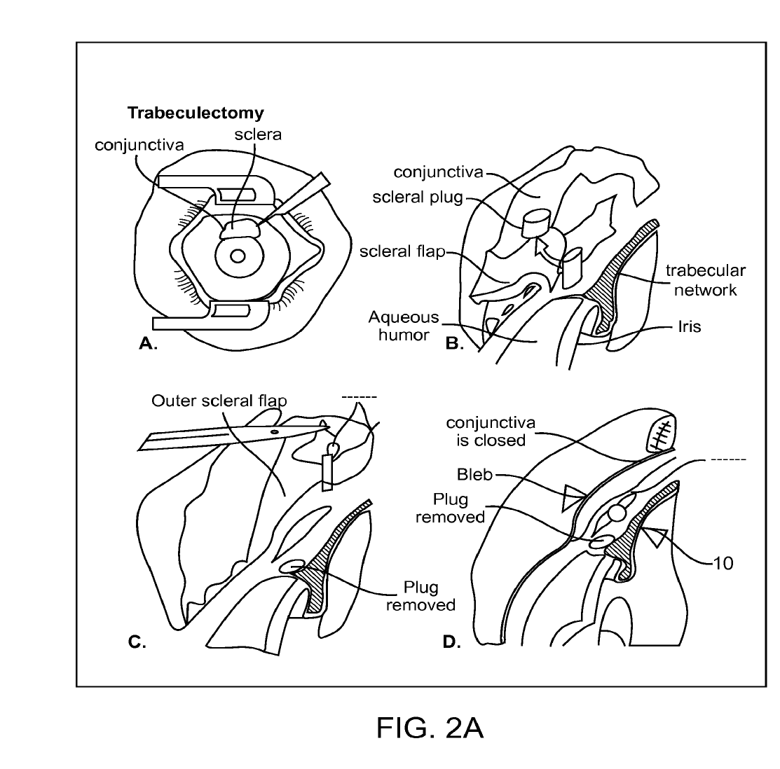

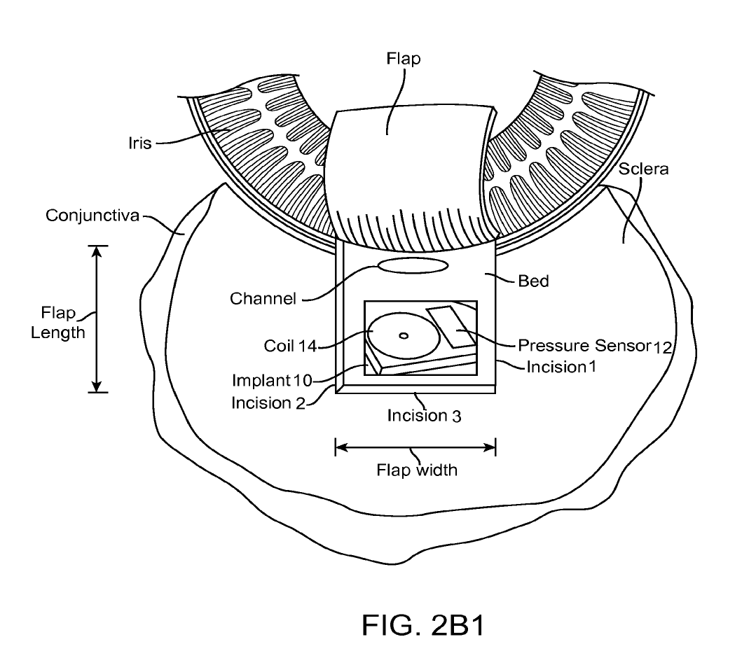

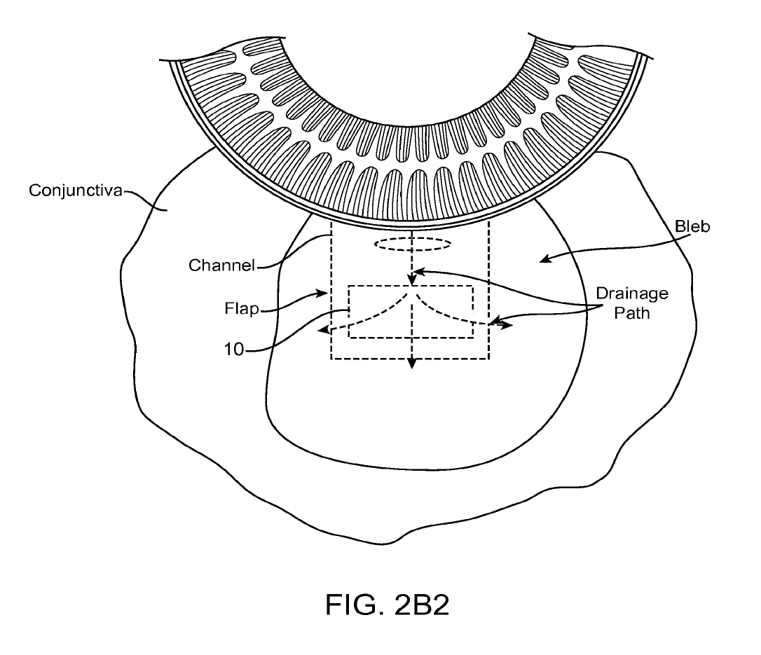

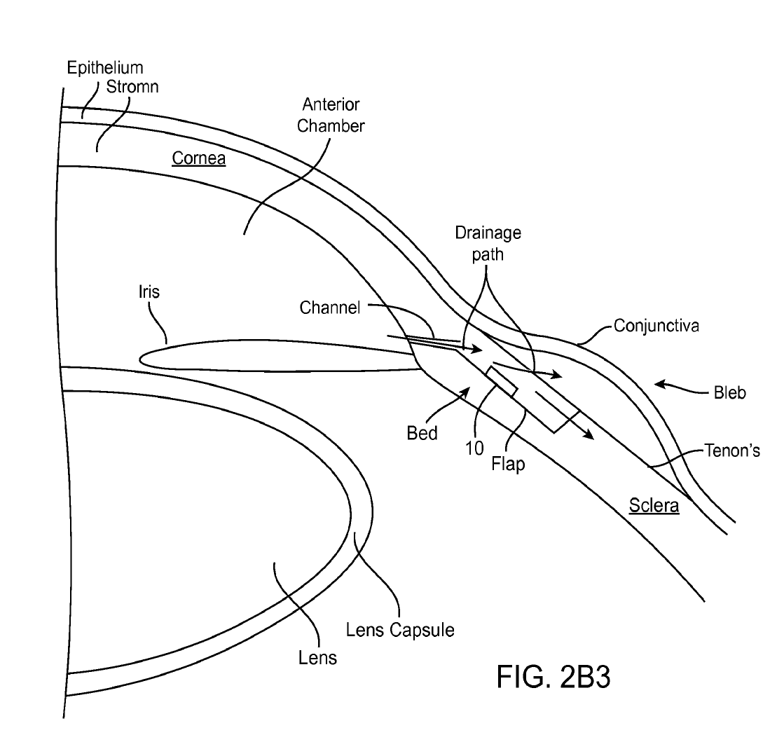

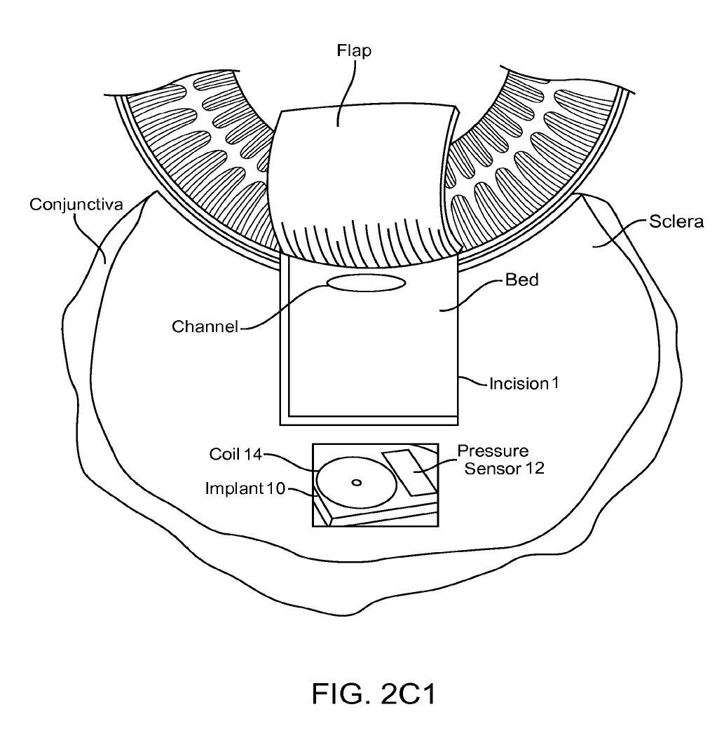

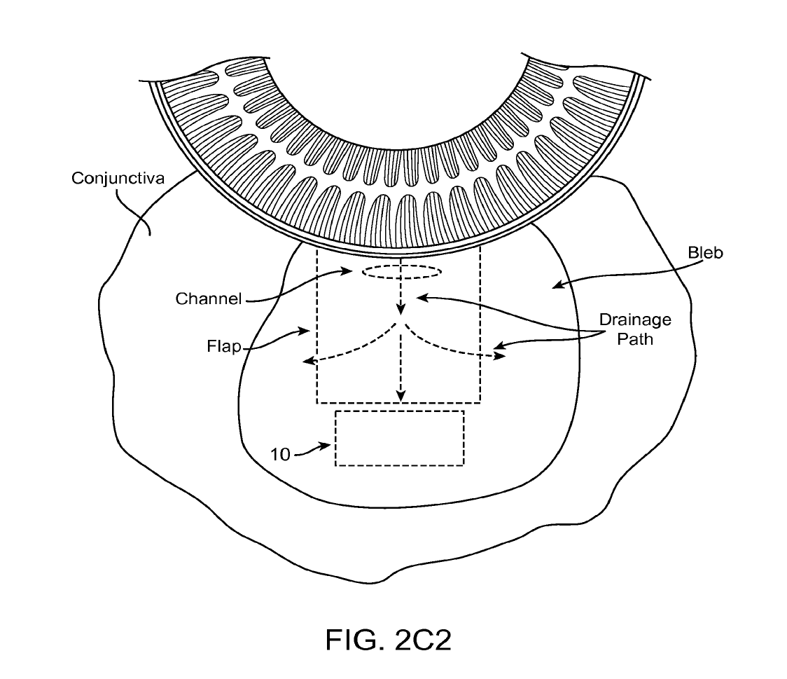

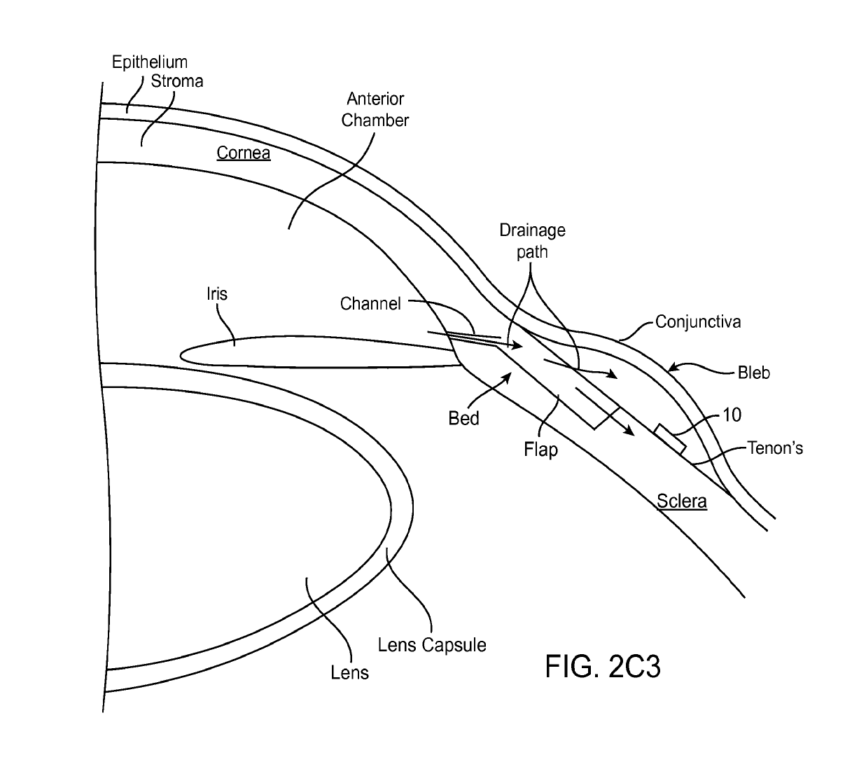

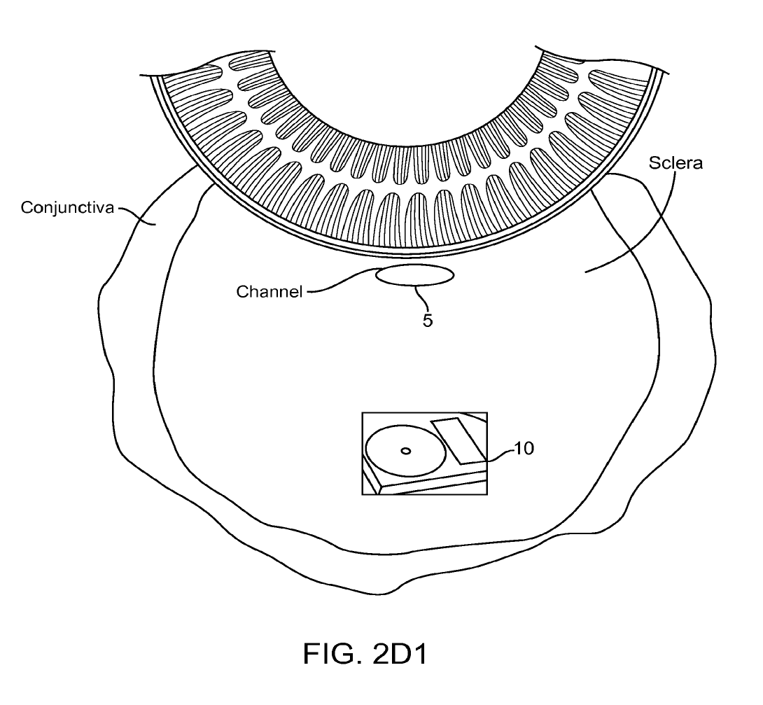

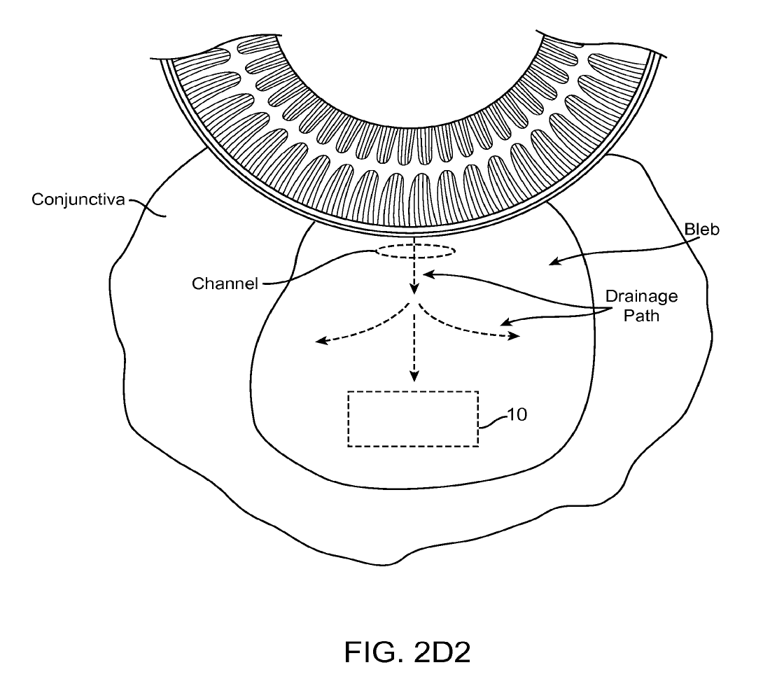

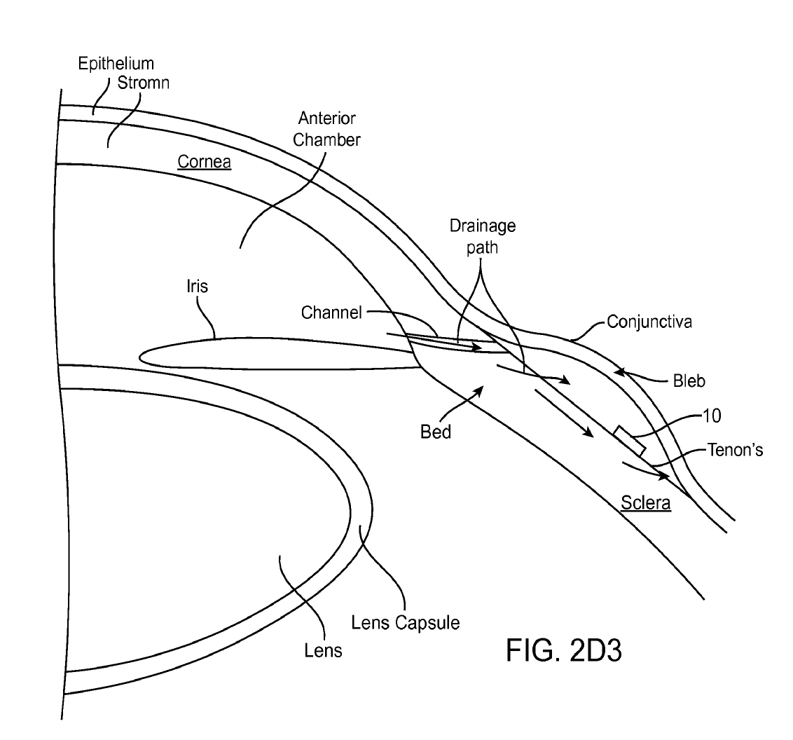

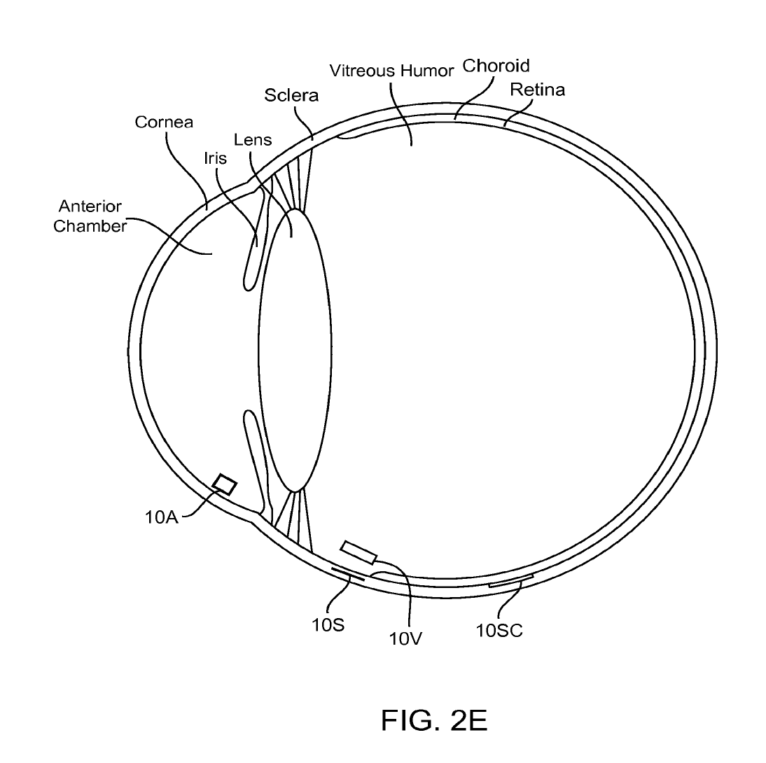

FIG. 2A shows placement of an implantable sensor device 10 along a surgically created tissue drainage path, in accordance with embodiments of the present invention. During a trabeculectomy, the patient's eyelids are retracted and the conjunctiva (outer layer) and sclera (white of the eye) are incised (A). A scleral flap is created and a filtration pathway is established (B) to allow the fluid (aqueous humor) in the eye to egress out of the anterior chamber, thus relieving pressure. The implant as described herein is positioned on the bed, and as indicated by the arrow. The scleral flap (C) and conjunctiva (D) are closed and sutured. A trabeculectomy and illustration similar to FIG. 2A and suitable for modification and use in accordance with embodiments described herein can be found at the online Encyclopedia of Surgery available on the Internet (http://www.surgicalencyclopedia.com and illustrated by GGS, Inc.)

FIGS. 2B1 to 2B3 show a trabeculectomy and an implantable sensor device 10 positioned on a scleral bed for covering with the flap. FIG. 2B1 shows the sensor positioned on a bed of the sclera with the flap lifted. The sensor can be placed on the bed and sutured in place so as to anchor the sensor device to the bed. FIG. 2B2 shows the flap positioned over the sensor device 10 post-surgically and the bleb. FIG. 2B3 shows a side profile view of the post-surgical eye as in FIG. 2B2. The conjunctiva is cut so as to form an opening to access the sclera. At least three incisions are made in the sclera so as to allow the flap to be lifted. For example a first incision, incision 1 and opposite incision, incision 2, can be spaced apart so as to define a width of the flap. A third incision, incision 3 can be made across incision 1 and incision 2, so as to define the length of the flap. The flap can then be lifted to expose the surface of the stromal bed. A channel can be formed so as to extend from the exposed surface of the stromal bed through the trabecular meshwork and into the anterior chamber of the eye. The implant can be positioned on the bed and covered with the flap, such that the implant can measure the intraocular pressure of the eye.

The implant comprises a pressure sensitive capacitor 12 and an inductor comprising a coil 14. The coil is coupled to the inductor so as to form an LC tank circuit, the resonant frequency of which is determined by the capacitance and the inductance. The resonant frequency changes in response to changes in capacitance due changes in pressure on the capacitive pressure sensor.

The implant 10 can be sized in many ways to fit under the flap. The flap may comprise a flap length and a flap width. The sensor may comprise a corresponding sensor length and a corresponding sensor width. Each of the corresponding sensor length and sensor width can be sized smaller than the dimensions of the flap length and the flap width. For example, the flap may comprise a length of about 8 mm and a width of about 8 mm, and the implantable sensor device may comprise a length of about 7 mm and a width of about 7 mm, such that the flap is sized for placement on the bed and under the flap. The implantable sensor device may comprise many lengths and widths, for example about 6 mm by 6 mm, 3 mm by 6 mm, 3 mm by 2 mm, or 2 mm by 2 mm. The implantable sensor device may comprise an area corresponding to the length and width within a range from about 3 mm² to about 6 mm². The implantable sensor device may comprise a thickness, and the thickness may comprise no more than about 1.0 mm, for example no more than about 0.5 mm or no more than about 0.3 mm, such that the sensor can be retained for an extended period.

The implant can be positioned in many ways to determine the IOP. The implant is generally positioned along the drainage path of the liquid from the aqueous humor, such that the success of the surgery can be monitored. The drainage path may comprise the drainage path of the liquid from the anterior chamber resulting from the trabeculectomy or the trabeculotomy, such as the channel that extends to the anterior chamber, the scleral bed, if present, and the bleb. For example, the drainage of liquid can separate the conjunctiva from the sclera so as to form a bleb along the drainage pathway. The implant can be positioned under the conjunctiva above the sclera and within the bleb, for example on the sclera near the channel extending to the anterior chamber. Alternatively, the sensor can be positioned along the drainage pathway on the bed directly over the channel such that the sensor is coupled directly to the anterior chamber with the channel. The implant can be positioned along the drainage pathway on the bed away from the channel, such that the sensor measures IOP with liquid coupled to the anterior chamber through the channel and extending between the underside of the flap and the upper side of the bed. The implant may also be placed on the bed such that the implant extends partially over the channel, for example with the capacitive sensor disposed over the channel and the coil positioned away from the channel.

FIGS. 2C1 to 2C3 show placement of a sensor as in FIG. 2B1 with a trabeculectomy, in which the sensor positioned on the sclera away from the flap and bed and such that the sensor is located within the bleb when the bleb forms. FIG. 2C1 shows the sensor positioned on the Tenon's capsule of the sclera away from the flap and bed. The conjunctiva has been opened with an incision to access the Tenon's capsule. FIG. 2C2 shows post-surgical eye with the flap positioned over the bed and the conjunctiva positioned over the flap and over the sensor device 10. FIG. 2C3 shows a side profile view of the post-surgical eye as in FIG. 2C2 with the sensor 10 positioned on Tenon's capsule of the sclera and within the bleb. The post-surgical drainage has formed the bleb such that the sensor is positioned within the bleb. The aqueous humor liquid of the anterior chamber drains along the drainage path extending along the channel and the sclera so as to form the bleb. The implant sensor device 10 may be positioned on an upper layer of the sclera comprising Tenon's capsule. Alternatively, the implantable sensor device 10 may be positioned below Tenon's capsule.

FIGS. 2D1 to 2D2 show placement of a sensor as in FIG. 2B1 with a trabeculotomy, in which the sensor positioned on the sclera such that the sensor is located within the bleb when the bleb forms. FIG. 2D1 shows the sensor positioned on the Tenon's capsule of the sclera away from the drainage channel. The conjunctiva is incised to access the sclera, and the channel extending from Tenon's capsule to the anterior chamber is formed so as to drain liquid and lower IOP. FIG. 2D2 shows the post-surgical eye with the drainage channel and the conjunctiva positioned over the sensor device 10 and the drainage path. FIG. 2D3 shows a side profile view of the post-surgical eye as in FIG. 2D2 with the sensor 10 positioned within the bleb. The implantable sensor device is positioned at a location of the eye that corresponds to a location of the bleb when formed, for example within about 8 mm of the drainage channel, in some embodiments directly over the drainage channel. The conjunctiva is positioned over the implant 10 and the incision of the conjunctiva may be sutured closed. The liquid drainage path, as indicated with arrows, extends from the anterior chamber, along the channel into the sclera. The liquid can travel along layers of the sclera to the bleb or between the conjunctiva and Tenon's capsule, and the liquid can drain outward so as to separate the conjunctiva from the sclera and form the bleb.

FIG. 2E shows locations and surgical placement of the implantable sensor 10 in accordance with embodiments. The implantable sensor device can be placed at one or more of the following locations of the eye: intracorneal, anterior chamber, anterior segment, posterior chamber, posterior segment, vitreous and vitreous cavity, sub-retinal space, suprachoroid, suprachoroidal space, subconjunctiva, episcleral, intrascleral, periocular, trabeculotomy sites, trabeculectomy sites, or cyclodialysis space. For example, implantable sensor 10 may comprise implantable sensor 10A placed in the anterior chamber of the eye. Alternatively or in combination, implantable sensor 10 may comprise implantable sensor 1 OS positioned in the sclera of the eye. Implantable sensor 10 may comprise implantable sensor 10 SC positioned in the suprachoroidal space.

System Components and Function

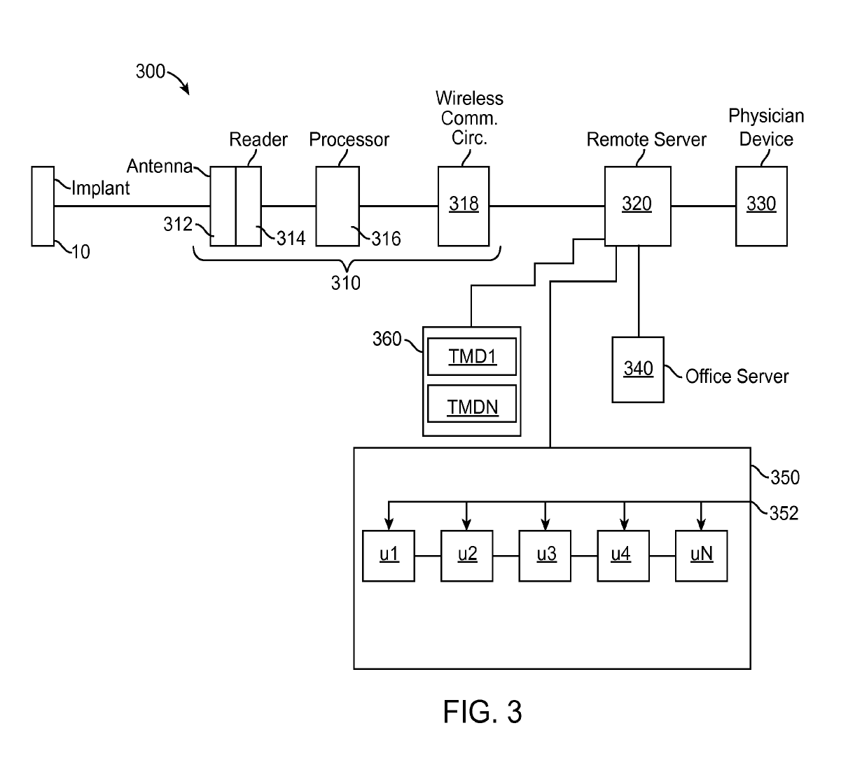

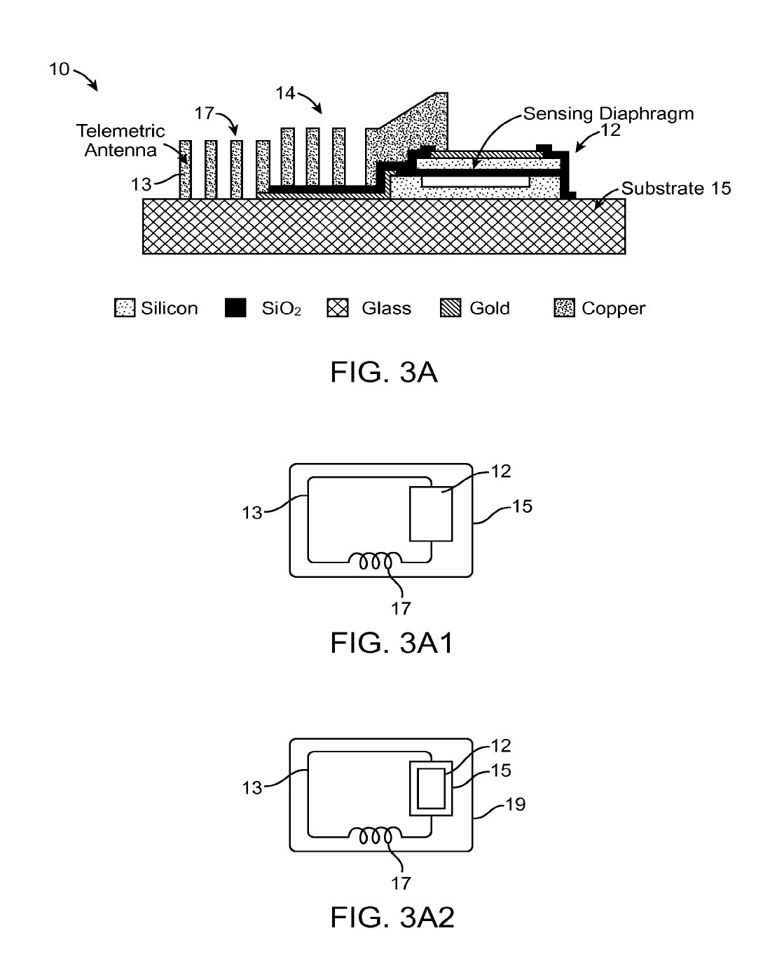

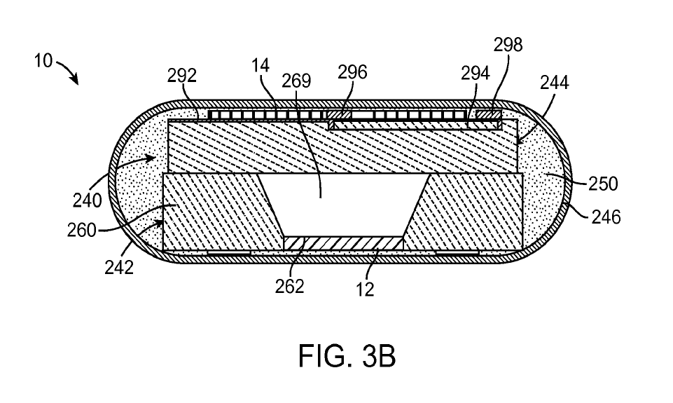

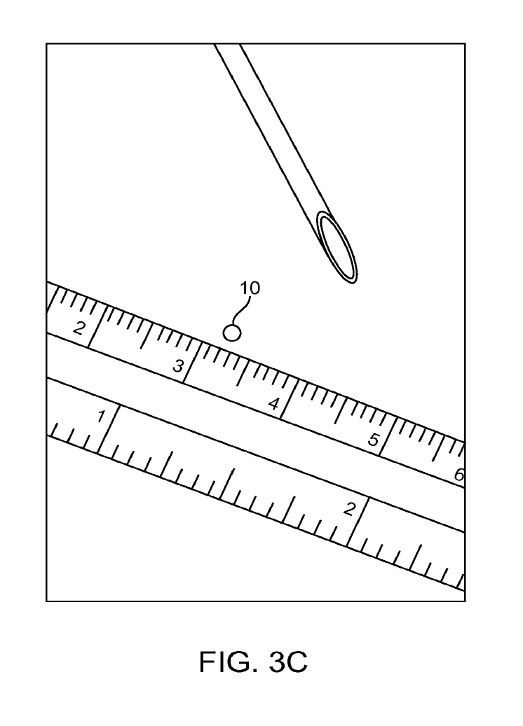

FIG. 3 shows components of a telemetry system 300 comprising the implantable sensor. The wireless communication based pressure sensing system may comprise several components. The implantable sensor 10 is configured to couple to an external reader 310, for example an antenna/reader, to determine the resonant frequency of the pressure sensitive capacitor and inductor circuit. The antenna/reader comprises an antenna 312 and reader circuitry 314 to determine the resonant frequency of the implanted sensor. The external reader 310 is configured to determine the patient IOP based on the directly measured pressure within the eye and the external atmospheric pressure. As atmospheric pressure can fluctuate approximately +/−10 mm of Hg and may also change with the elevation of the patient, the accuracy of the patient IOP reported to the physician and patient can be improved substantially by determining the reported IOP based on the IOP measured directly with the implanted pressure sensor and the atmospheric pressure external to the eye.

The external reader 310 can be configured in many ways to determine the IOP of the patient based on the directly measured IOP and the atmospheric pressure. For example, the external reader 310 may comprise an atmospheric pressure sensor to determine the IOP reported to the physician and the patient based on the IOP measured directly with implanted sensor and the local atmospheric pressure. Alternatively or in combination, the external reader 310 may have two way communication with an external weather site to determine the atmospheric pressure from the external site. For example, the external site may comprise a local weather station or web site having a corresponding internet address, and the atmospheric pressure where the patient is located can be determined based on one more of postal zip code, latitude and longitude, or global positioning system coordinates. The external reader 310 may comprise circuitry to determine the location of the patient and use the patient position in formation to determine the pressure where the patient is located based on meterological weather information. The global positioning coordinates of the patient can be determined in many ways, for example with location based on a cellular phone connection of the external reader 310 or based on GPS circuitry of the reader 310.

Atmospheric pressure associated with weather can fluctuate slowly and on the order of +/− about 10 mm of Hg, such that correction of measured patient IOP based on commercially available meteorological information can be sufficient to provide accurate determination of the patient IOP when combined with the directly measured IOP. Also, by determining the location of the patient, fluctuations in atmospheric pressure associated with the elevation where the patient is located can determined and used to determine the patient IOP. For example, the IOP reported to the physician and patient can be determined by subtracting the barometric pressure at the location and elevation of the patient from the directly measured IOP to determine the corrected IOP reported to the physician and patient. The elevation of the patient can be determined based on the location of the patient, for example when the patient is located at a city near sea level or a city in the mountains. The rate of change in patient location can also be used, for example when the patient flies and location changes quickly.

The adjusted IOP (AIOP) for patient reporting and can be determined in many ways based on the directly measured internal IOP and externally measured atmospheric pressure. For example, the adjusted IOP (ΔIOP) may comprise a differential IOP determined by subtracting the external atmospheric pressure (ATP) from the internally measured IOP (IMIOP) with the equation (AIOP)=(ΔIOP)=(IMIOP)−(ATP).

Although a calculation is shown, the adjusted IOP can be determined in many ways, for example with a look up table stored in a processor.

The antenna/reader is coupled to a processor 316 comprising a computer readable medium having instructions of a computer program embodied to determine the intraocular pressure, for example with a look up table, in response to the resonant frequency. The data can be stored on a personal computer of the at least one processor, for example a laptop. The at least one processor can be coupled to the Internet with wired or with wireless communication circuitry 318 and transmit the patient data to a server 320 located remote from the patient.

The external antenna/reader 310 may comprise a hand-held ambulatory device comprising the atmospheric pressure sensor, the processor 316 and the wireless communication circuitry such that the patient can transmit measurement data with the wireless communication circuitry. For example, the wireless communication circuitry may comprise one or more of Wi-Fi circuitry or cellular circuitry, such that the patient user can measure and transmit data to the central server when the patient is mobile. The handheld ambulatory external reader 310 may comprise circuitry similar to hand held communication devices such as pagers and smart phones, for example the iPhone™ or the Blackberry™ smart phones. The handheld external reader 310 may comprise instructions of a computer readable program embodied on a tangible medium to determine the IOP reported to the physician based on the IOP measured directly with implanted sensor and the atmospheric pressure. For example, the atmospheric pressure can be determined based on the location and elevation of the patient and local barometric pressure, as described herein.

The remote server 320 may comprise data from many patients and comprise instructions of a computer program embodied on a programmable memory, such that the data from many patients can be combined and analyzed. For example, the server may comprise a data center where data are analyzed and physicians can share patient data. Alternatively or in combination, the patient data can be transmitted to a treating physician for evaluation of the patient. For example, the data can be transmitted to a server 340 located at the treating physicians office. The data can also be transmitted to the physician with wireless cellular communication, for example with to a handheld physician communication device 330 such as a pager, iPhone™ smart phone, or Blackberry™ smart phone, such that the physician can evaluate the status of the patient and may adjust treatment of the patient accordingly.

The system 300 may comprise a processor system, and the processor system may comprise two or more of the processor located with the patient, the remote server, the server located at the physician office, or the hand held physician communication device 330.

The hand held communication device 330 can be configured such that the physician can transmit treatment instructions for patient treatment so as to close the loop of the treatment for the patient, for example with changes to medication or requesting a patient examination. The remote server comprises processor comprising a computer readable medium having instructions of a computer program embodied thereon so as to store patient data with a database. The remote server may also forward treatment instructions from the physician device 330 to the patient device 310.

The instructions from the handheld physician communication device allow the physician to direct patient treatment. For example, the physician can instruct the patient to come in for a visit, for example to assess the status of the patient need for additional surgical intervention. The physician may adjust the patient medication, for example increase the patient medication. The physician may set a target IOP for the patient based on the clinical assessment of the patient. Some patient who have lost vision can be more sensitive to IOP than those who have not, such that the physician may set the target IOP for a patient with vision loss lower than a patient who has not lost vision. For example, the physician can set the target IOP for a patient with vision loss at 12 mm Hg, and the target IOP for a patient with no vision loss at 21 mm Hg. The physician assessment of patient vision loss can be determined in many ways, for example with one or more of visual fields testing or the cup to disk ratio which is known measurement to assess the progression of glaucoma. The above treatment instructions may comprise menu selections of hand held physician device 330 that can be selected and forwarded to the hand held patient reader device 310.

The handheld communication device 330 may comprise a processor comprising a computer readable medium having instructions of a computer program embodied thereon so as to store and display patient data for diagnosis and treatment, for example data received from the server. The server located at the physician office may comprise a processor comprising a computer readable medium having instructions of a computer program embodied thereon so as to store patient data with the database. The remote server may comprise the server at the physician office.

The remote server 320 can be configured to communicate with processors of a community 350 of online users. The community 350 of online users may comprise a plurality of processors 352. The plurality of processors 352 may comprise, for example, a first user processor U1 of a first user, a second user processor U2 of a second user, a third user processor U3 of a third user and a fourth user processor U4 of a fourth user and an Nth user processor UN of an Nth user, for example a one millionth user. The online community 350 may comprise patients monitored with the implanted sensor device and friends, family members and care givers of the patients. The community of user may be connected with an online community social networking site comprising a virtually community. For example the online community may comprise Facebook users.

The remote server 320 can be coupled to a community of remote online physicians 360 who can compare data and who can provide telemedecine to members of the online community 350. The community of remote online physicians can practice telemedecine with a patient, for example a patient of the community of users. The treating physician and physician device 330 may comprise a member of the community of remote online physicians 360. Each physician has access to a processor comprising a tangible medium having computer readable instructions stored thereon, for example a smartphone, a tablet computer, a notebook computer or a desk computer. For example a first processor TMD 1 comprising a smart phone may be used by a first physician and a second processor TMD2 comprising a notebook computer may be used by a second physician.

The remote server 320 can control communication and access of the patient data, and may be configured to display information on the displays of the online community 350 and the processors of the community of remote online physicians. The remote server 320 can receive commands from the physician and transmit the treatment commands to the hand held external reader 310. For example, the physician can prescribe a target IOP for the patient based on the physician's evaluation of the patient, and the customized physician prescribed target IOP can be transmitted to the hand held external reader 310. The handheld external reader may comprise instructions of a computer program such that a message is transmitted to the treating physician, for example an email, when the patient IOP exceeds the customized prescribed target IOP. Alternatively or in combination, the remoter server may comprise instructions to transmit a message to the physician when the patient IOP exceeds the physician prescribed IOP for the patient.

Wireless Pressure Sensor.

FIG. 3A shows components of an implantable sensor as in FIGS. 2A and 2B. The capacitive pressure sensor is connected to the spiral inductor to create a the LC resonant tank circuit. The tank circuit lends comprises a miniature wireless sensor. The sensor can have dimensions sized so such that the area of the sensor positioned within the bleb or on the bed comprises an area within a range from about 3 to 6 mm², and can be shaped and sized for implantation under the sclera flap, for example a standard cut of 8×8 mm, and for positioning next to or on top of the channel of the filtration pathway. At the thickest portion of the sensor, the sensor may have a height of no more than 0.5 mm, for example, although the sensor can be thicker, or thinner, depending on the placement location.

The pressure sensors may comprise many of types of known biocompatible pressure sensors having sized for placement in the bed or within the bleb.

The sensor may comprise a micro-electro-mechanical system (MEMS) and can be fabricated with known methods. The coil may be fabricated on the same substrate as the pressure sensor (1-chip), or it alternatively it can be separate and attached to the pressure sensor (2-chip). For example, the sensor may comprise a single chip sensor supported with a substrate 15, such as a glass substrate. Alternatively, sensor 10 may comprise a hybrid sensor having the MEMS pressure sensor supported on a flexible substrate such as a flex PCB, for example a polyimide flex PCB, and the telemetric antenna may comprise a substantially single loop antenna deposited on the flex PCB with traces of an electrical conductor so as to define the loop antenna.

The sensor may have a plurality of layers deposited on substrate 15. A layer of conductive silicon semiconductor can be deposited on the glass substrate 15 and shaped with lithography and etching so as to form a lower side of the capacitor. A layer of gold can be deposited over the silicon and glass so as to form a lead extending from the from the lower side of the capacitor to center of the coil. A dielectric layer, for example Si0₂, can be deposited over the gold to insulate the antenna from the lead and separate the lower side of the capacitor from the upper side. A layer of conductive silicon semiconductor can be deposited on the dielectric layer opposite the lower side of the capacitor and shaped to form the upper side of the capacitor. The upper side of the capacitor may comprise a sensing diaphragm that bends with pressure so as to decrease spacing of the first side of the capacitor from the second side such that the capacitance increases when pressure increases. A layer of conductor, for example gold, can be deposited on the second side of the capacitor comprising the pressure sensing diaphragm, and the conductor can be shaped to couple to the coil 14 comprising the telemetric antenna 13 and turns 17 of the coil 14. A conductor, for example copper, can be deposited at least partially over the dielectric layer and sensing diaphragm such that the lower side of the capacitor is coupled to the inner portion of the coil and the upper portion of the capacitor comprising the sensing diaphragm is coupled to the outer portion of the coil.

The pressure sensor may be calibrated for the elevation of the location where the patient lives, and can have an average pressure and frequency corresponding to the pressure at the elevation of the location where the patient lives.

FIG. 3A1 shows the implantable sensor 10 in which the coil 14 comprising a substantially single loop telemetric antenna 13 coupled to a second a second coil having the inductive turns 17 so as to provide the resonant frequency based on the pressure of the sensor. The coil 14 comprising the substantially single loop telemetric antenna 13 and the second coil having the inductive turns 17 can be formed on the substrate 15.

FIG. 3A2 shows the implantable sensor comprising a MEMS pressure sensor locate on a support 19 such as a flex PCB support, such that the implant 10 comprises a hybrid implantable sensor. The coil 14 may comprising the substantially single loop telemetric antenna 13 coupled to the second a second coil having the inductive turns 17 so as to provide the resonant frequency based on the pressure of the sensor. The coil 14 comprising the substantially single loop telemetric antenna 13 and the second coil having the inductive turns 17 can be formed on the support 19, for example with traces of metallic conductor extending along support 19. In many embodiments the second coil having inductive turns 17 may comprise a toroidal inductor, for example, placed on support 19 and coupled to substantially single loop telemetric antenna 15 with pads disposed on support 19.

Packaging.

FIG. 3B shows packaging of an implantable sensor, in accordance with embodiments of the present invention. To protect the MEMS pressure sensor with wireless telemetry from corrosion, the implant device 10 as described herein may be coated or encapsulated in a soft biocompatible polymer such as polydimethlysiloxane (PDMS). The sensor can read pressure from all directions as, a result of its compliant enclosure 246, which is filled with a conformable material 250 such as liquid, viscous material, or gel (e.g., silicone, saline or other biocompatible material). This allows pressure to be uniformly exerted on the pressure sensor 12, such that pressure can be sensed from forces on a side opposite the pressure sensor. For example, the implanted device can be positioned such that the pressure sensor is located on a first side of the implant opposite a second side of the implant, and the implanted device can measure pressure when the second side of the implant is positioned and oriented so as to contact liquid of the channel or bleb and the first side of the implant with the sensor is positioned and oriented away from the channel or bleb and in contact with tissue.

The implantable device may comprise packaging with fenestrations, or holes, such that the implanted device can be anchored to tissue with sutures, when appropriate.

The transducer assembly 240 may comprises a pressure sensor 12 and a telemetric device comprising coil 14. The transducer assembly 240 may comprise the capacitive pressure sensor and inductor on the substrate as described above. The transducer assembly 240 can be encased in a compliant enclosure 246 that is responsive to external pressure. The compliant enclosure 246 may comprise a balloon-like sac made of a biocompatible material that surrounds the transducer assembly 240. Alternatively, the compliant enclosure 246 may comprise a gel, gelatin, or film of biocompatible materials.

The compliant enclosure 246 can be filled with a liquid (or a gel) 250, such as silicone, saline, or other suitable material, that is biocompatible. The properties of the liquid 250 allow the liquid to transmit pressure exerted against the compliant enclosure 246 uniformly against the sensing element of the pressure sensor 12, while isolating the electrical components and circuitry of the transducer assembly 240 from corrosive media.

The illustrated pressure sensor 12 may comprise a known configuration and can be made using known micromachining processes, micro fabrication processes, or other suitable MEMS fabrication techniques. Pressure sensors of this type are commercially available from Motorola, Inc. of Schaumburg, 111. and TRW Novasensor of Fremont, Calif. It should be understood that many pressure sensors meet the biocompatibility and size requirements and may be used.

The illustrated pressure sensor 12 may comprise a piezoresistive device, and many types of pressure sensors, such as a piezoelectric and capacitive sensors, can be substituted. The pressure sensor 12 may comprises a substrate 260, a sensing diaphragm 262, a plurality of patterned resistors, and a plurality of bond pads, two of which can be associated with each of the resistors.

The substrate 260 may have upper and lower surfaces and can be made of silicon, but could alternatively be made of another suitable material. The substrate 260 has a well region 269 that extends between the upper and lower surfaces and that can be formed using a conventional microfabrication and bulk micromachining processes including lithography and etching. The sensing diaphragm 262, which extends across the well region 269, can also made of silicon and is defined by the lithography and etching processes. The resistors and the bond pads can be formed from a metal or polysilicon layer that is deposited, patterned, and etched in a known manner on the lower surface 268 of the substrate 260. The resistors could also be formed by doping the silicon using boron, phosphorus, arsenic, or another suitable material to render a region of the silicon with an appropriate conductivity and polarity to create junction-isolated piezoresistors. As will be apparent to those skilled in the art, other methods, such as SIMOX, wafer bonding, and dissolved wafer approaches, could also be used. The resistors can be positioned along the edges of the sensing diaphragm 262 to detect strain in the sensing diaphragm caused by pressure differentials. The resistors could alternatively be positioned in another region of high or maximum strain in the sensing diaphragm 262.

The packaging may be shaped and sized for easy insertion and fixation. For example, the packaging may comprise a first side having a first outer surface and a second side having a second outer surface opposite the first side, in which the first side and the second side extend substantially along a plane, such that the device can be implanted between layers of the sclera. The outer portion comprising the perimeter can be rounded, so as to decrease point localization of forces to the scleral tissue and so as to couple smoothly to the tissue.

Additionally; the packaging containing the device can be further encapsulated with therapeutically active agents to alter wound healing and control infection, thus improving the success rate of the glaucoma filtration surgery (GFS), as the failure rate of GFS can be as high as 25%. For example, the therapeutically active agent may comprise Mitomycin C, or other antimetabolite for example, so as to decrease scar formation.

Delivery Tool.

FIG. 3C shows a delivery tool for placement and an implantable sensor as in FIG. 2A. A ruler is shown for scale, and the diameter of the implant device can be about 2 mm. The packaged wireless pressure sensor can be placed with a cannula, or other delivery tool that protects the sensor assembly and facilitates insertion. For example, the packaged wireless pressure sensor can be placed into the interior of the bleb, under the sclera flap, or within an incision. The inner lumen of the delivery tool can be sized to receive the implant device. For example, the implant device may comprise a maximum cross sectional size of no more than about 2 mm, for example a diameter of 2 mm, such that the device can be implanted with delivery tool having a lumen diameter of about 2 mm.

Antenna/Reader.

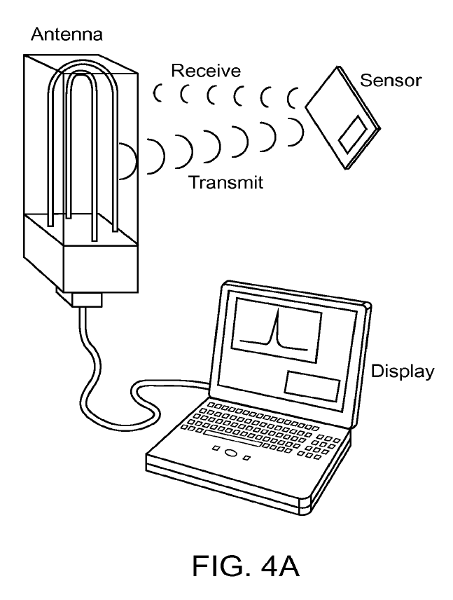

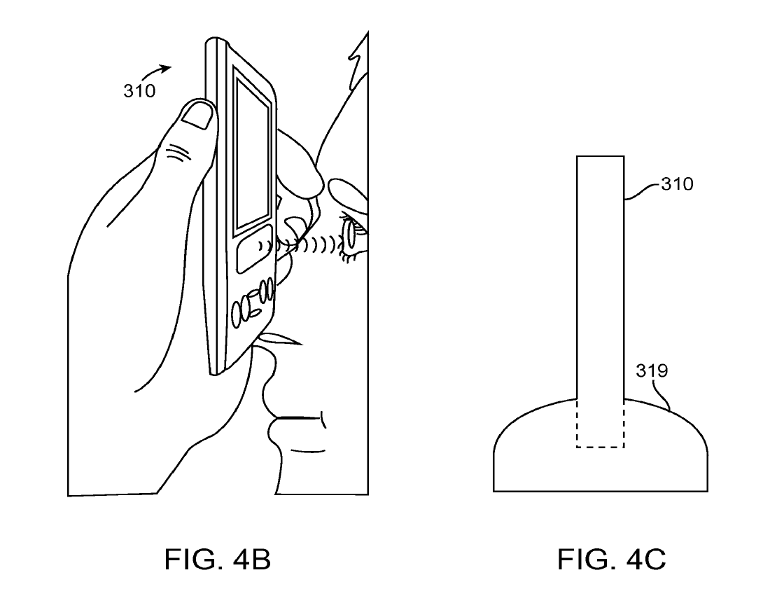

FIG. 4A shows components of an external reader 310 comprising an antenna/reader and processor coupled to the antenna/reader to determine the IOP. The radio-frequency probe comprises circuitry to emit a radio frequency signal with the antenna so as interrogate the tank circuit of the implanted sensor device, such that the resonant frequency of the LC tank circuit can be determined. As the resonant frequency changes with pressure, the IOP measured with the sensor can be determined based on the resonant frequency. The reader can house the electronics and software, and may comprise a processor having a computer readable medium having instructions of a computer program embodied thereon so as to be used as a data collection, reporting and analysis platform, for example data mining to determine the presence of pressure spikes and trends. The processor can be programmed to measure the IOP and predetermined intervals or predetermined times, or both. The processor can be coupled to the Internet and the servers as described above.

FIG. 4B shows a hand held external reader 310 comprising an antenna reader with components similar to the antenna reader as in 4A. The hand held data reader may comprise the antenna, circuitry to determine the resonant frequency, and circuitry similar to a smart phone such as an iPhone™, such that the hand held reader can measure, store and transmit patient data.

FIG. 4C shows a docking station 319 to receive the hand held antenna reader as in 4B. The docking station can be configured to charge the external reader 310 and may be used to transfer data from the external reader to the remote server. For example, the docking station 319 may comprise serial communication such as a universal serial bus (USB) communication to download measurement data from the external reader 310. The docking station 319 may comprise communication circuitry to transmit the data from the docking station to the remote server, for example one or more of wireless circuitry or wired circuitry.

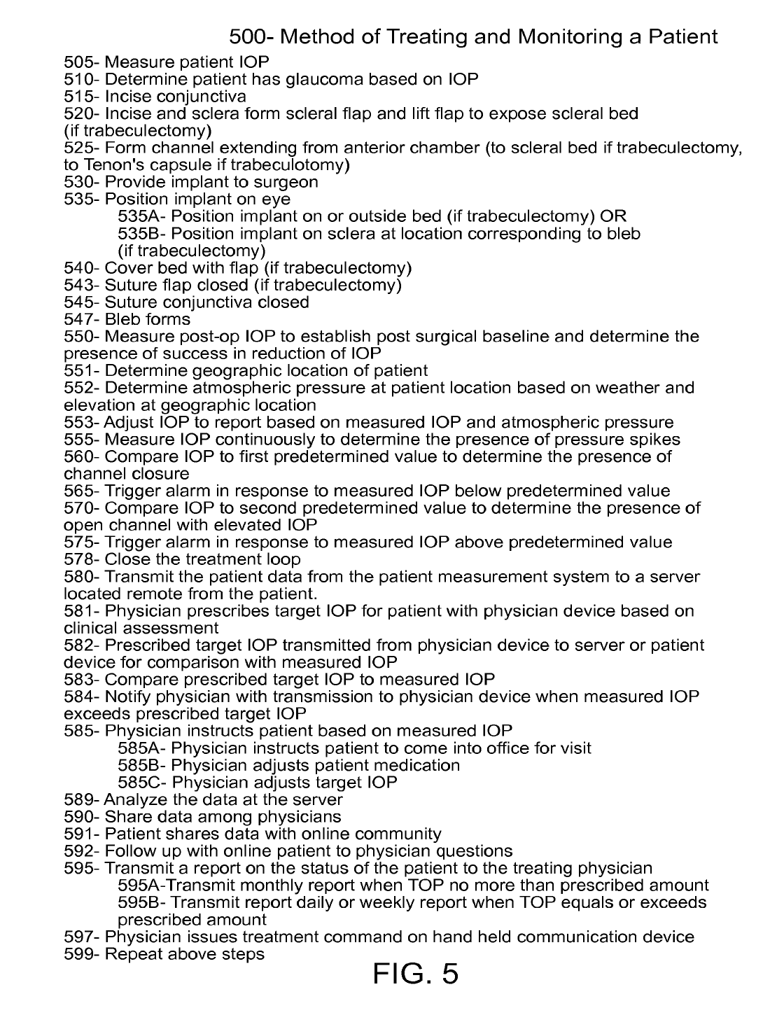

FIG. 5 shows a method 500 of treating and monitoring a patient. The method 500 can be used with surgery such as a trabeculectomy or a trabeculotomy, and a person of ordinary skill in the art will recognize appropriate adaptations of method 500 for use with a selected surgery. A step 505 measures patient IOP. A step 510 determines that the patient has glaucoma based on the IOP, for example a physician diagnosis that may include other patient testing such as visual fields and optical coherence tomography scans of the retina. The physician may prescribe drugs as an intermediate step, and the physician may subsequently determine that the patient needs surgery to treat glaucoma. A step 515 incises the conjunctiva. A step 520, for example with a trabeculectomy, incises the sclera and forms a scleral flap and lifts the flap to expose a scleral bed, as described above. A step 525 forms a channel extending from the anterior chamber. The channel can extend to the scleral bed with a trabeculectomy, and the channel can extend to Tenon's capsule with a trabeculotomy. A step 530 provides an implant, as described above, to the surgeon. A step 535 positions the implant on the eye. For example, a step 535A positions the implant on the bed of a trabeculectomy and sutures the implant to the bed of the trabeculectomy. Alternatively, a step 535B position the implant on the sclera, for example on Tenon's capsule, at a location corresponding to the bleb, such that the implant is located within the bleb. Step 535B can be used with a trabeculectomy, for example. The implant may be injected into a pocket formed under the conjunctiva. A step 540, for example with a trabeculectomy, covers the bed with the flap. A step 543 sutures the flap closed, for example with a trabeculectomy, and the implant may be sutured in position with sutures that extend through the fenestrations on the implant. A step 545 sutures the incision in the conjunctiva closed.

A step 550 measures post-op IOP to establish a post surgical baseline and determine the presence of a reduction in IOP. A step 551 determines the geographic location of patient. A step 552 determine atmospheric pressure at patient location based on weather and elevation at geographic location. A step 553 adjusts IOP to report based on measured IOP and atmospheric pressure.

A step 555 measures IOP at regular intervals, for example hourly, and may measure IOP continuously, so as to determine the presence of pressure spikes. A step 560 compares the IOP to a first predetermined value to determine the presence of channel restriction, for example closure. A step 565 triggers and alert, for example an alarm, in response to the measured IOP below the predetermined value. The predetermined value may comprise, for example 12 mm of mercury (Hg). A step 570 compares the IOP to a second predetermined value to determine the presence of an open channel with an elevated IOP. A step 575 triggers an alarm in response to the measured IOP above the predetermined value. A step 578 closes the treatment loop based on the directly measured IOP. The treatment loop can be closed in many ways, for example with an adjustment of patient medication, or adjustment to a valve of an implanted glaucoma shunt device similar hydrocephalus valve. A step 580 transmits the data from the patient measurement system to a server located remote from the patient.

At a step 581 a physician prescribes a customized target IOP for the patient with the physician device based on the physician's assessment of the patient. At a step 582, the prescribed customized target IOP is transmitted to from the physician device to one or more of the server or the patient device for comparison with the measured IOP. At a step 583, the physician prescribed customized target IOP is a compared to the measured patient IOP. At a step 584, the physician is notified when the measured patient IOP exceeds the prescribed target IOP, for example with an email from the server to the physician device.

At a step 585, the physician instructs patient based on the measured IOP. For example, the physician may select instructions from a menu. At a step 585A, the physician instructs patient to come into office for visit. At a step 585B, the physician adjusts patient medication. At a step 585C, the physician adjusts target IOP. The physician can identify each of these instructions and select one or more these steps from a menu so as to instruct the patient.

A step 589 analyzes the data at the server, for example with data modeling to determine statistical trends. As the communication from the patient to the physician and back may comprise two-way communication routed through the central server, the data available can be useful. The data analysis may comprise mining the patient data with instructions of a computer program embedded on a tangible medium of the remote server. A step 590 shares data among physicians, for example with a registry of patient data for analysis, and physicians of the online physician community can share data with each other.

At a step 591, patients share information and data online, for example with the online community. For example, a family member or care giver can follow up on the care of an elderly patient who shares data with the family member or care giver. At a step 592, a member of the online community can ask questions of physicians, for example the treating physician of the online physician community.

A step 595 transmits a report on the status of the patient to the treating physician, for example to a computer system at the physician's office and/or to a hand held communication device such as an iPhone™ or Blackberry™ or a pager. The report can be transmitted based on the directly measured patient IOP. For example, at a step 595A the report can be generated monthly when the directly measured patient IOP remains within normal limits and at or below the physician prescribed IOP. However, at a step 595B, the treatment report can be generated daily, every few days, or weekly, when the directly measured IOP equals or exceeds the physician prescribed target or when the directly measured IOP exceeds the range of the pre-determined upper and lower limits.

At a step 597, the physician issues a treatment command on the hand held communication device, for example an adjustment to the patient medication.

At a step 599, the above steps are repeated.

It should be appreciated that the specific steps illustrated in FIG. 5 provide a particular method of treating and monitoring a patient, according to an embodiment of the present invention. Other sequences of steps may also be performed according to alternative embodiments. For example, alternative embodiments of the present invention may perform the steps outlined above in a different order. Moreover, the individual steps illustrated in FIG. 5 may include multiple sub-steps that may be performed in various sequences as appropriate to the individual step. Furthermore, additional steps may be added or removed depending on the particular applications. One of ordinary skill in the art would recognize many variations, modifications, and alternatives.

The processor system as described above can be configured to implement many of the steps of method 500. For example, the processor system may comprise a computer readable medium having instructions of a computer program embodied thereon to implement many of the steps of method 500, for example steps 550 to 599.

### EXPERIMENTAL

A person of ordinary skill in the art can conduct experimental studies based to determine empirically, the location of the sensor in the bed and predetermined values of the measured pressure to determine the presence of channel closure. Such studies can be conducted with an animal model, for example rabbits, and clinical studies with patients may also be conducted.

Experimental Testing with Rabbits

The below described rabbit testing shows successful real time direct measurement of IOP in the rabbit animal model. Similar measurements can be made with human glaucoma surgery as described herein, for example with an implantable sensor device sized for placement in the surgical drainage pathway such as a 3 mm by 3 mm sensor. The demonstrated direct measurement of intraocular pressure of the aqueous humor and transmission of the electromagnetic pressure signal through the corneal tissue to the external reader corresponds substantially to the direct measurement of IOP with a sensor located in the surgical drainage pathway and tissue transmission through the flap to the external reader as described herein. For example, the transmission of the measured EM signal through the cornea and aqueous humor disposed between the implanted sensor and external reader comprises a transmission distance through tissue that is at least as much as the tissue transmission distance through the flap of sclera and conjunctiva for the measurement of IOP along the tissue drainage pathway as described herein.

TABLE I
Specifications for Implantable Sensor.

| | |
|---|---|
| Range | 0-50 mm Hg (alternatively 0-60 mm Hg) |
| Mean | 18-25 mm Hg |
| Resolution | 1 mm Hg |
| Working distance | 2-4 cm (alternatively 0-6 cm) |
| Sensor Chip Size | 6 × 6 mm |
| Shape | Square chip, with circular external |
| | packaging |
| Accuracy (absolute pressure) | +/−2 mm Hg |

Table I shows exemplary specifications for the implanted device in accordance with embodiments as described herein. The range can be from about 0-50 mm Hg for testing, although other ranges can be used such as 0-60 mm Hg. The mean sensor reading can be within a range from about 18-25 mm Hg. The resolution of the sensor reading can be about 1 mm Hg. The working distance from the measurement probe to the eye can be from about 2-4 cm, although other ranges can be used such as 0-6 mm such that the probe can touch the eyelid. The sensor chip size can be about 6×6 mm square. The shape of the implantable sensor may comprise a square chip with circular external packaging. The absolute accuracy of the sensor device can be +/−2 mm Hg. Three (3) chip sensors as described herein comprising 6×6 mm Sylgard® encapsulated biosensors were selected for this experiment.

New Zealand White rabbits (NZW): Surgical Procedure:
Three (3) 4-5 kg New Zealand White (NZW) rabbits (2 females, 1 male) were sedated with IM Ketamine HCL (40 mg) and Xylazine HCL (2 mg). A retrobulbar 2% (10 mg) (0.5 ml) was administered to the left eye of 2 animals and to the right eye of the third rabbit. The lids were expose w speculum and topical 0.5% properacaine HCL and 5% Betadine drops were instilled twice before surgery. A limbal-based conjunctival flap was fashioned superiorly and a limbal groove incision was made with a #15 Bard Parker scalpel. The anterior chamber was entered at 12 o/c with a knife-needle and the wound was enlarged with Castroviejo corneal scissors 3-9 o/c superiorly. The corneal flap was reflected forward with Colibri forceps to exposed the anterior chamber. The implantable sensor was then inserted into the anterior chamber and positioned in-place centrally with McPherson forceps and iris spatula. The wound was then closed with interrupted vertical mattress 6-0 chromic sutures and the conjunctival flap was closed with running 6-0 chromic suture. At the end of the procedure the anterior chambers were observed to have reformed without evidence of any wound leak. Tobrex® (tobramycin 0.3%) was instilled in the superior and inferior cul-de-sac. Rabbits were examined daily by gross and slit-lamp. Topical Tobrex ointment was administered after each examination. The implanted eyes of each animal were judged to be clinically free of post-surgical inflammation by day 5.
In-vivo IOP sensing with the implanted chip sensor:
The left eye of one implanted NZW (#15) was selected for in vivo IOP sensing approximately 6 days post-implantation.

### Instrumentation Overview:

An implantable chip sensor as described above was implanted in the anterior chamber of the eye of a rabbit, approximately days before measurements were made. The sensors are designed to shift their resonant frequency in response to absolute pressure. The resonant frequency of the implanted sensor is measured by an external reader, which utilizes a custom antenna, a network analyzer, and a custom software application running on a PC.
Prior to implantation, the pressure-frequency response of the sensor is characterized, and is saved in a calibration file on the PC. After implantation, the software uses the network analyzer to measure the center frequency of the sensor, then uses the calibration file to convert frequency to pressure.

### Instrumentation Setup:

Setup materials & equipment:

Orthogonal antenna

### Vector Network Analyzer (VNA, HP 8753C)

VNA—antenna interface hardware

Directional coupler, 10 dB, (Olektron A2655-03)

4 db N type attenuator

Antenna tuner & balun (custom)

### Splitter (Minicircuits ZFSCJ-2-1-S)

Receiver amplifier (+34 dB, Qbit QB-164-LH)
Lowpass filter (Minicircuits BLP-100+

Coaxial cables with SMA terminations

Clamp-on ferrites (3ea, Fair-rite 0443665806)

Antenna compensation coil (custom)

Lab stand (for holding antenna)

### Laptop PC (Dell D610)

Data acquisition and frequency-pressure conversion software (Custom, AcuMEMs)
GPIB-USB adapter (National Instruments)
Tonometer (for external IOP measurement): iCare tonometer TA01 i
Setup procedure:
Place the antenna on a non-metallic table (the surface may not contain a conductive loop—e.g. a metal frame around the perimeter of the table).
“Null” the antenna
Adjust the orthogonal antenna position to minimize cross coupling (minimize background signal on the VNA)

Adjust the tuner to achieve nominal “flatness” in the 40-50 MHz region

Compensate the effect of the patient body
Place the patient, with the un-implanted eye near the antenna

Adjust the compensation coil position to again minimize the background

Start the pressure-logging software on the PC, and load the sensor file.

### Procedure:

The patient was anaesthetized to minimize rapid movements during data acquisition

The implanted eye was moved close (within 5 mm) to the antenna.

The software recorded data during the following protocol

Confirm detection of the sensor in VNA manual mode

Run acquisition for several minutes to establish a baseline
Use soft-tipped swab to externally apply pressure to the sclera of implanted eye Minimize any repositioning of the patient's head when applying pressure

Maintain applied pressure, with as much stability as possible, for a few minutes

Gently remove the swab

Continue to record data for several more minutes

Repeat the above pressure application (discretionary)

### Data:

Sensor characterization before implantation:
Date of characterization: XX/XX/XXXX
Nominal center frequency (at local atmospheric pressure):

Conditions:

encapsulated in silicone elastomer comprising Sylgard™ 529

placed inside of a small PVC pressure chamber

container placed directly on side of antenna cover

sensor approx. 5 mm from antenna cover

Pressure-Frequency Characterization:

Measured scale factor: −0.06612 MHz/psi×0.019337 psi/mm Hg=−0.0012786 MHz/Hg

Null frequency (0 PSI gauge): 45.12 MHz Sensor in-vivo measurements:
Sensor characterization after implantation:

### Date, Time: XX/XX/XXXX

Local pressure: 29.78 in. Hg
Animal description: Rabbit, female, approx. 4 kg, approx. 12 wks old
Implantation site: anterior chamber of left eye, sensor #XXX-Y-ZZ

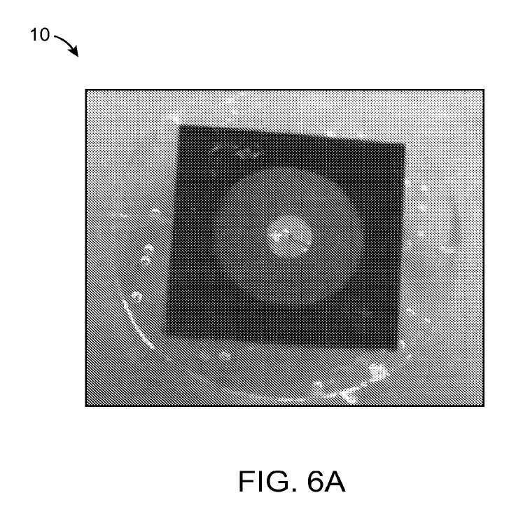

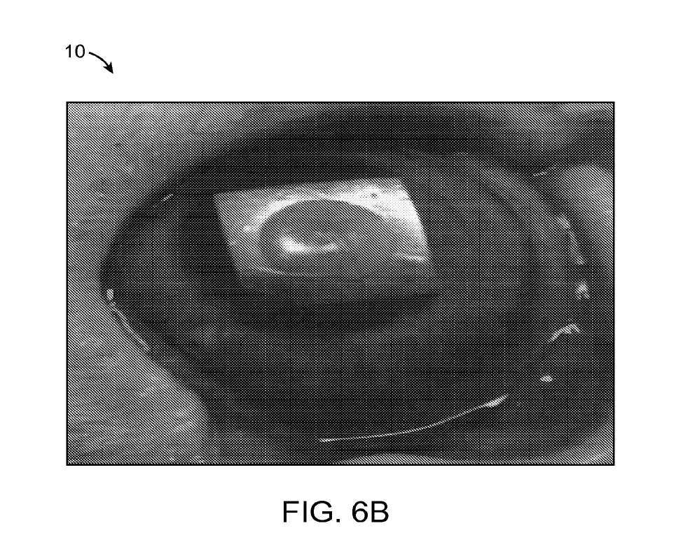

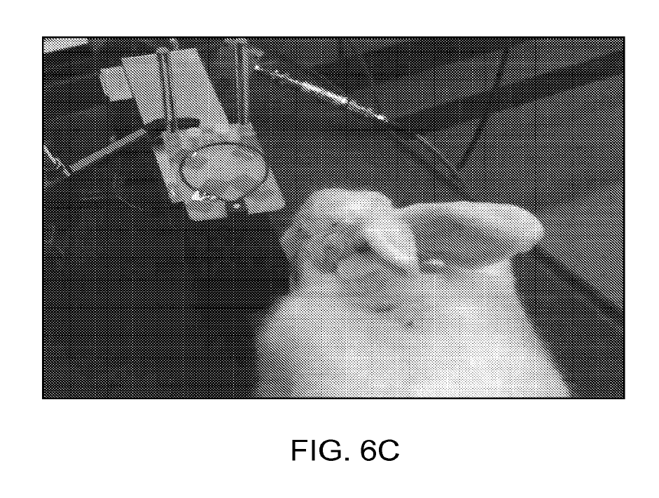

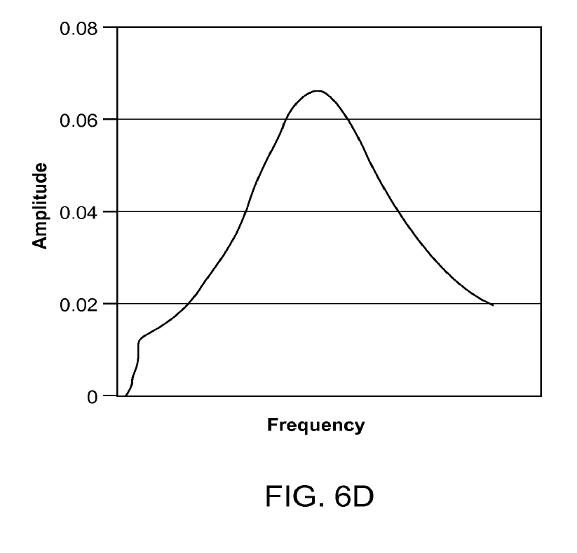

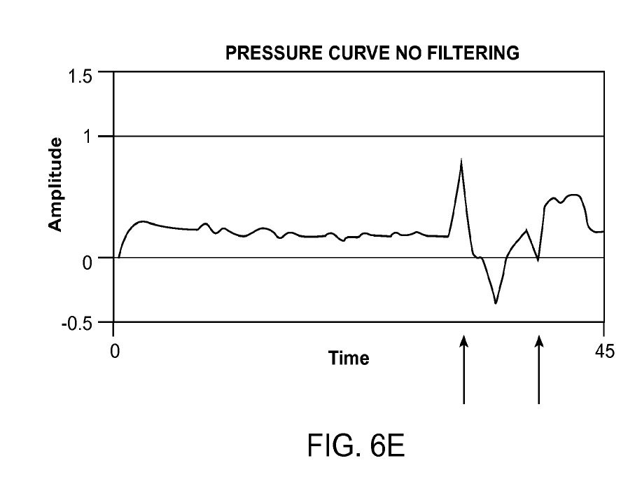

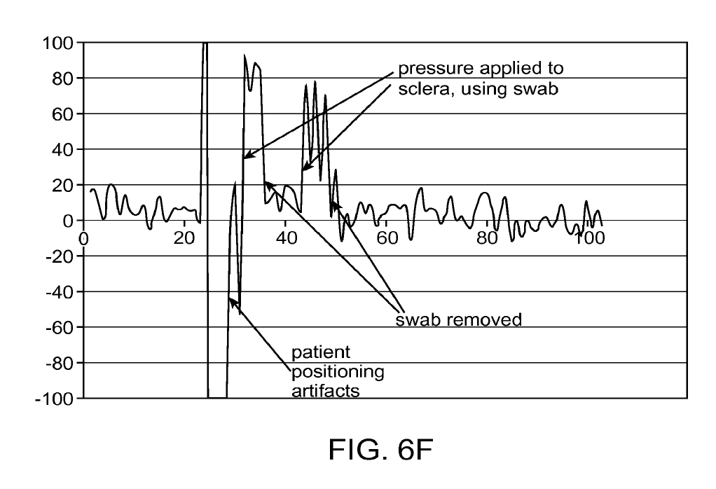

FIG. 6A shows an implantable sensor for direct measurement of IOP prior to placement in an eye of a rabbit. The implantable sensor device comprises a chip sensor comprising a capacitive sensor and coil embedded in a complaint transparent enclosure as described above. The substrate supporting the coil comprises an approximately 6 mm by 6 mm square having the circular coil and capacitor disposed thereon. The substrate and circuitry comprise a thickness of about 250 um and the total thickness with the complain enclosure comprises about 500 um. The circular complaint enclosure comprise a circular perimeter and a diameter of about 7 mm, such that the complaint enclosure extends around and covers the corners of the substrate with a clearance of about 0.5 mm on each corner.

FIG. 6B shows the experimentally tested implantable sensor as in FIG. 6A implanted in the anterior chamber of an eye of a rabbit. The sensor is implanted under the cornea and above the pupil and can be readily seen in the eye of the rabbit.

FIG. 6C shows a rabbit positioned near an antenna reader with the sensor implanted as in FIGS. 6A and 6B. The head of the rabbit is positioned near the telemetry coil of the reader for direct measurement of IOP.

FIG. 6D shows a distribution of sensor signals and a peak of the distribution for IOP measured directly with the rabbit positioned near the sensor as in FIG. 6C. The peak of the distribution corresponds to a measured IOP of about 3 mm of Hg. This direct measurement of IOP compared well with a known veterinary tonometer (Icare™ VET) commercially available from Icare of Finland.

FIG. 6E shows pressure shifts of IOP measured directly over time with the rabbit positioned near the sensor reader as in FIG. 6C. The amplitude signal corresponds to the IOP of the eye and is measured in units that correspond to mm of Hg. The direct IOP measurement is shown for about 45 seconds. The amplitude of the signal (arbitrary units) goes from −0.5 to 1.5, and 0 corresponds to an IOP of 0. The signal from about 5 seconds to about 30 s has an amplitude of about 0.2 to 0.3 and corresponds to an IOP of about 3 to 4 mm of Hg. To evaluate the real time response of the implanted sensor, the eye was touched with a Q-tip™ cotton swap at about 30 seconds (shown with first vertical arrow), and the measured IOP elevated substantially. The Q-tip™ was removed and the measured IOP went negative. The Q-tip™ cotton swap was applied again at about and the pressure similarly elevated (shown with second vertical arrow). The directly measured IOP increase was verified with the external tonometer that showed an IOP of about 13 mm Hg. Upon removal of the cotton swab, the IOP decreased to about 3-4 mm Hg at 45 seconds.

TOP measurements using iCare tonometer:
Initial IOP of unimplanted eye: 6 mmHg
Initial IOP of implanted eye: 3 mmHg
Pressure change test on implanted eye:

3 mmHg (initial pressure)

16 mmHg (with swab applying pressure to sclera)
21 mmHg (2nd measurement with swab applying pressure to sclera)
Recorded Data from “Reader”:
Fc used for measurements: 45.0 MHz (center of scan range—not necessarily 0 psi frequency)
Span used for measurement: 5.0 MHz
Changes in the center frequency of the sensor are produced by changes in absolute pressure. To resolve relative (gauge) pressure, local atmospheric pressure can be accounted for. The data in the graph was manually adjusted for offsets in MS Excel. Time is not scaled in the graph, but each point represents approximately 15 seconds between acquisitions. Changes in center frequency can also result from other environmental effects, including presence of nearby metal, the patient body, and motion artifacts. It can be important to minimize motion artifacts, to avoid placing the antenna near metal objects, and to compensate for the effects of the patient body (using the compensation coil). The large excursions around samples 22-30 in FIG. 6F are motion artifacts, for example. The signal from the sensor was readily detectable at approximately 45 MHz. During the testing, the frequency shifted readily (downward) with the application of pressure on the sclera (using a swab). The corresponding pressure shifts, as measured by the sensor and reader, were larger than similar readings taken subsequently with the tonometer.

FIG. 6F shows IOP measured directly over time with calibration for the rabbit positioned near the sensor reader as in FIG. 6C. Direct measurement of IOP is shown to about 100 seconds, and the calibrated measured IOP is shown in mm of Hg. The measurements show increases in measured IOP when the swap if applied to the sclera, and decreases in the directly measured IOP when the swab is removed. Although some measurement artifacts are shown, the data can be filtered to remove these artifacts, for example with digital filtering. Also, lower TOP after applied pressure is consistent with recovery of the eye and ocular tissues.

### CONCLUSIONS

The implantable, wireless sensors are capable of measuring intra-ocular pressure changes. Further testing can characterize the absolute and relative accuracies of the sensor and reader measurements over the range, for example simultaneous external indirect measurements and internal direct measurements.

Although the above experiments show good signal measurements, one of ordinary skill in the art can make improvements. For example, it may be helpful to strengthen the signal from the sensor and enhance its sensitivity to pressure. Also, the sensor and reader may be configured so as to have less sensitive to environmental effects.

While the exemplary embodiments have been described in some detail, by way of example and for clarity of understanding, those of skill in the art will recognize that a variety of modifications, adaptations, and changes may be employed. Hence, the scope of the present invention shall be limited solely by the appended claims and the equivalents thereof.

What is claimed is:

1. A method of implanting a pressure sensor in an eye of a patient to measure IOP, the eye having an internal tissue and an internal liquid, the method comprising:
providing an implant device comprising a pressure sensor, an inductive coil, and a compliant material, the compliant material disposed over the pressure sensor to define an outer portion of the implant device; and
placing the outer portion of the implant device in contact with the internal tissue of the eye such that the outer portion of the implant device is coupled to the tissue of the eye to measure the IOP.

2. The method of claim 1, wherein the implant device is placed at one or more of intracorneal, anterior chamber, anterior segment, posterior chamber, posterior segment, vitreous and vitreous cavity, sub-retinal space, suprachoroid, suprachoroidal space, subconjunctiva, episcleral, intrascleral, periocular, trabeculotomy sites, trabeculectomy sites, or cyclodialysis space.

3. A method of implanting a pressure sensor in an eye of a patient, the eye having a sclera and an anterior chamber, the method comprising:
providing an implant device comprising a pressure sensor, an inductive coil, and a compliant material, the compliant material disposed over at least a portion of the pressure sensor to define an outer portion of the implant device;
forming a channel extending from the anterior chamber at least partially into the sclera to drain liquid from the anterior chamber, the sclera defining at least a portion of the channel; and
placing the outer portion of the implant device in contact with the sclera such that the outer portion of the implant device is coupled to the anterior chamber with the liquid drained from the anterior chamber.

4. The method of claim 3, wherein the pressure sensor comprises a transducer and the complaint material.

5. The method of claim 3, wherein the pressure sensor comprises a capacitive pressure sensor.

6. The method of claim 5, wherein the coil comprises a substantially single loop antenna coupled to a second coil having plurality of turns.

7. The method of claim 5, wherein the implant device comprises a substrate and wherein the capacitive pressure sensor and the inductive coil comprise micro electromechanical structures formed on the substrate.

8. The method of claim 3, wherein the eye comprises a conjunctiva and wherein a bleb disposed between the sclera and the conjunctiva is formed to drain the liquid and wherein the pressure sensor is positioned between the sclera and the conjunctiva at a location disposed within the bleb.

9. The method of claim 3, further comprising forming a flap comprising a partial thickness of the sclera to expose a scleral bed, the scleral bed having a cross sectional size and a depth sized to receive the implant device, and wherein the channel extends from the anterior chamber to the bed to drain liquid from the anterior chamber.

10. The method of claim 9, wherein the channel extends from a distal end opening into the anterior chamber to a proximal end opening into the bed of the sclera.

11. The method of claim 9, wherein the implant device is positioned on the bed to measure the pressure of the anterior chamber and the flap is positioned over the implant device.

12. The method of claim 11, wherein at least the outer portion of the implant device is positioned on the proximal end of channel to measure the intraocular pressure.

13. The method of claim 11, wherein the implant device comprises at least one fenestration sized to receive a suture and wherein the implant device is anchored to the sclera with the suture.

14. The method of claim 3, wherein the implant device comprises an outer surface and the compliant material is shaped to define the outer surface and wherein the implant device is positioned such that at least a portion of the outer surface is coupled to the liquid to measure the pressure.

15. The method of claim 3, wherein the complaint material extends over the pressure sensor to measure pressure along a 360 degree perimeter.

16. The method of claim 3, wherein the pressure sensor comprises a transducer and the complaint material, and wherein the compliant material extends over the transducer such that the implant device responds to pressure along an outer surface of the complaint material.

17. The method of claim 3, wherein the implant device comprises a first side and a second side and the pressure sensor comprises a capacitive pressure sensor comprises a diaphragm disposed on the first side and wherein the complaint material extends over the first side and the second side such that the implant device responds to pressure along the second side.

18. The method of claim 17, wherein the implant device measures the pressure of the channel with the second side disposed toward the channel and the first side having the capacitive sensor and the diaphragm disposed away from the channel.

19. The method of claim 17, wherein the compliant material extends substantially around the first side and the second side and contains a conformable material to transmit pressure from the second side to the first side.

20. The method of claim 3, wherein the implant device comprises a length, a width and a thickness and wherein the bed comprises a corresponding length width and depth each less than the length, the width and the thickness of the implant device such that the bed is sized to receive the implant device.

21. The method of claim 20, wherein the length and the width of the implant device are sized such that the implant device is retained between the flap and the bed.

22. The method of claim 20, wherein the length and the width of the implant device are greater than the thickness of the implant device.

23. The method of claim 20, wherein the thickness comprises no more than about 0.5 mm.

24. The method of claim 20, wherein the length and the width are sized such that a corresponding area comprises no more than about 50 mm.

25. The method of claim 20, wherein the length and the width are sized such that a corresponding area comprises no more than about 10 mm.

26. The method of claim 20, wherein the length and the width are sized such that a corresponding area comprises no more than about 5 mm.

27. The method of claim 20, wherein the length comprises no more than about 7 mm and the width comprises no more than about 7 mm.

28. The method of claim 20, wherein the length comprises no more than about 3 mm and the width comprises no more than about 3 mm.

29. The method of claim 20, wherein the length comprises no more than about 2 mm and the width comprises no more than about 2 mm.

30. The method of claim 3, further comprising measuring the pressure with the pressure sensor based on a resonant frequency of the inductor and the capacitive sensor.

31. The method of claim 30, wherein the measured pressure is compared to a predetermined value to determine closure of the channel.

32. The method of claim 31, wherein an alert is triggered in response to the measured pressure below the predetermined value.

33. The method of claim 32, wherein the predetermined value corresponds to about 12 mm of mercury.

34. The method of claim 30, wherein an IOP reported to the patient is determined based on the measured IOP and an atmospheric pressure.

35. The method of claim 30, wherein an IOP reported to the patient is determined based on the measured IOP and a location of the patient.

36. The method of claim 3, wherein the implant device comprises fenestrations size to receive sutures and wherein the implant device is anchored to the sclera with sutures extending through the fenestrations.

37. The method of claim 3, wherein the implant device is anchored to a scleral bed of a trabeculectomy with sutures extending through fenestrations of the implant device.

38. A method of monitoring a patient, the method comprising:
measuring an internal pressure of the eye with a pressure sensor disposed within the eye; and
determining an atmospheric pressure;
determining an IOP of the patient based on the atmospheric pressure and the internal pressure.

39. A method of monitoring a patient, the method comprising:
determining a location of the patient; and
determining an IOP of the patient based on the location of the patient.

40. A method of treating glaucoma of an eye of a patient, the eye having an IOP, a sclera and an anterior chamber, the method comprising:
providing an implant device comprising a pressure sensor, an inductive coil, and a compliant material, the compliant material disposed over at least a portion of the pressure sensor to define an outer portion of the implant device;
forming at least one channel extending from the anterior chamber at least partially into the sclera to drain liquid from the anterior chamber, wherein a tissue drainage pathway forms in response to formation of the at least one channel;
placing the outer portion of the implant device at a tissue location disposed along the tissue drainage pathway such that the outer portion of the implant device is coupled to the anterior chamber with the liquid drained from the anterior chamber to measure the IOP.

41. An apparatus for measuring an intraocular pressure of an eye of a patient, the eye having and an internal tissue and a liquid disposed therein, the apparatus comprising:
an implant device comprising a pressure sensor, an inductive coil, and a compliant material, the compliant material disposed over at least a portion of the capacitive pressure sensor to define an outer portion of the implant device, wherein the implant device is configured to measure the intraocular pressure when the outer portion of the implant device is placed against the tissue and coupled to the liquid.

42. An apparatus for measuring an intraocular pressure of an eye of a patient, the eye having anterior chamber with a liquid disposed therein, the apparatus comprising:
an implant device comprising a pressure sensor, an inductive coil, and a compliant material, the compliant material disposed over at least a portion of the capacitive pressure sensor to define an outer portion of the implant device, wherein the implant device is configured to measure the intraocular pressure when the outer portion of the implant device contacts liquid drained from the anterior chamber.

43. The apparatus of claim 42, wherein the implant device is configured with the conformable material and a capacitive pressure sensor to measure the intraocular pressure in response to pressure around an outer boundary of the implant device.

44. The apparatus of claim 43, wherein the coil comprises a substantially single loop antenna coupled to a second coil having plurality of turns.

45. The apparatus of claim 42, further comprising a hand held reader configured to couple to the implant device to determine the intraocular pressure when the outer portion contacts the sclera.

46. The apparatus of claim 45, further comprising the hand held reader is configured to determine the intraocular pressure of the patient based on an atmospheric pressure determined in response to a location of the patient.

47. An apparatus to measure intraocular pressure of an eye of a patient, the eye having an anterior chamber and a sclera, the apparatus comprising:
implantable means for measuring the intraocular pressure.
